(19) United States
(12) Patent Application Publication
(10) Pub. No.: US 2025/0258176 A1
(43) Pub. Date: Aug. 14, 2025

(54) METHOD FOR CLASSIFYING CANCER PATIENTS INTO APPROPRIATE HEPATOCELLULAR CARCINOMA TREATMENT GROUPS AND COMPOUNDS FOR TREATING THE PATIENT

(71) Applicant: SINGAPORE HEALTH SERVICES PTE. LTD., Singapore (SG)

(72) Inventors: Suk Peng CHEW, Singapore (SG); Salvatore ALBANI, Singapore (SG); Kah-Hoe Pierce CHOW, Singapore (SG); Lu PAN, Singapore (SG)

(73) Assignee: SINGAPORE HEALTH SERVICES PTE. LTD., Singapore (SG)

(21) Appl. No.: 19/074,447

(22) Filed: Mar. 10, 2025

Related U.S. Application Data
(62) Division of application No. 16/768,001, filed on May 28, 2020, now Pat. No. 12,276,664.

(51) Int. Cl. G01N 33/574; A61K 51/12

(52) U.S. Cl. CPC G01N 33/57438; A61K 51/1251; G01N 33/57492

(57) ABSTRACT

A method for the prognosis of response to treatment for a patient suffering from cancer, the method comprising: measuring the expression of at least one immune marker in a leukocyte sample taken from the patient with cancer; classifying the patient sample into (i) sustainable responders (SR) to selective internal radiation therapy (SIRT) or (ii) transient/non-responders (TR/NR) to (SIRT) based on expression of the at least one immune marker in relation to a predetermined value and treating sustainable responders (SR) to SIRT or SIRT or a composition comprising SIRT and an immunotherapy. In a preferred embodiment, the method is for the prognosis of response to treatment of a Hepatocellular carcinoma (HCC) patient comprising detecting the co-expression of PD-1 or Tim-3 with CCR5; or expression of PD-1, Tim-3, CXCR6, or combinations thereof in a leukocyte sample taken from the patient.

### CROSS REFERENCE TO RELATED APPLICATIONS

This application is a divisional application of application Ser. No. 16/768,001 filed on May 28, 2020, which is a national phase of International Application No. PCT/SG2018/050585 filed on Nov. 29, 20218, which claims the priority to Singapore Application No. 10201709924T filed on 30 Nov. 2017, the contents of which are incorporated herein by reference.

### FIELD OF INVENTION

The present invention relates to systems and methods for classifying cancer patients into treatment groups to prognosticate and to predict response to specific cancer treatments, specifically radiation therapy and in particular selective internal radiation therapy (SIRT) treatment alone or in combination with other therapies including immunotherapy.

### BACKGROUND TO THE INVENTION

The following discussion of the background to the invention is intended to facilitate an understanding of the present invention. However, it should be appreciated that the discussion is not an acknowledgment or admission that any of the material referred to was published, known or part of the common general knowledge in any jurisdiction as at the priority date of the application.

In 2015 cancer caused 15.7% of human deaths. A large portion of these fatalities include cancers that were diagnosed late and were at a stage that they could not be resected and/or had developed a hyper-vascularized mass around the cancer. A hyper-vascularized mass around a cancer receives its blood supply predominantly through newly formed branches of existing arteries such as the hepatic artery in the case of Hepatocellular carcinoma (HCC). A common treatment for hyper-vascularised cancers is to embolize the newly formed branch. Selective internal radiation therapy (SIRT) (also called radioembolization (RE) is one form of therapy whereby radioactive coated microspheres are delivered directly into the hyper-vascularised cancer via trans-arterial catheter under radiologic guidance. This precise mode of delivery spares the non-malignant organs as radiation therapy is only delivered through the branches supplying to the tumors and the radiation from the microspheres destroys the tumor cells within a limited area of emission while sparing the healthy tissues.

Hepatocellular carcinoma (HCC) is a highly malignant disease, and the second most common cause of cancer-associated deaths worldwide (Ferlay, et al. Int J Cancer 136, E359-386 (2015).). The most effective therapeutic options for HCC are tumor resection or liver transplantation, but these are limited to early stage disease (Bruix, and Sherman, Hepatology (Baltimore, Md.) 53, 1020-1022 (2011).). The majority of the patients who have locally advanced disease are treated with loco-regional therapies such as transarterial chemoembolization or Yttrium-90 (Y90) radioembolization (RE), also-known-as selective internal radiation therapy (SIRT) (Raza, and Sood, World Journal of Gastroenterology: WJG 20, 4115-4127 (2014).).

The liver is generally sustained as an immunosuppressive microenvironment. The main reason the liver has evolved in this way is that blood from the arterial circulation and the intestines enter the liver, where toxins and gut-derived microbial products are captured and eliminated. To prevent aberrant immunity in response to continual pathogen exposure, the liver has a unique system of immune regulation. Hepatocytes contribute to the liver's inherent tolerogenicity by priming naïve T cells in the absence of co-stimulation, resulting in defective cytotoxicity and clonal deletion. This suggests that immunotherapy particularly intrahepatic T cell priming would not be useful in treating HCC. Further, the liver processes toxins and as such is generally less susceptible to chemotherapeutic agents.

Yttrium-90 (Y90)-radioembolization (RE) significantly regresses locally advanced hepatocellular carcinoma (HCC) and delays disease progression. Despite its effectiveness, the immunological impact of Y90-RE, which elicits a sustained therapeutic response, is not well understood. Y90-RE has been shown to elicit a disease-control by tumor-downstaging and delayed disease progression (Kallini, et al. Advances in Therapy 33, 699-714 (2016).). The half-life of the Y90-isotope is ˜64.2 hours, but maximal clinical response, i.e. tumor regression and decrease in serum alfa-feto protein, is only seen 3-to-6-months after treatment (Salem, et al.

Gastroenterology 138, 52-64 (2010).). The mechanisms that underlie this delayed, yet long-lasting anti-tumor effect remain elusive.

There is a need to understand who will respond to treatments to ameliorate at least one of the problems mentioned above.

### SUMMARY OF THE INVENTION

It is an object of the present invention to provide systems and methods for classifying cancer patients into treatment groups to prognosticate and to predict response to specific cancer treatments, specifically whether radiation therapy will be an appropriate cancer treatment, in particular selective internal radiation therapy (SIRT) treatment alone or in combination with other therapies including immunotherapy.

Accordingly, an aspect of the invention provides an in vitro method for the prognosis of response to treatment for a patient suffering from cancer, the method comprising:
measuring expression of at least one immune marker in a leukocyte sample taken from patient with cancer;
classifying the patient sample into (i) sustained responders (SR) to selective internal radiation therapy (SIRT) or (ii) transient/non-responders (TR/NR) to (SIRT) based on the expression of the at least one immune marker in relation to a predetermined value.

Another aspect of the invention provides a system for prognosticating a response to treatment for a patient suffering from cancer, the system comprising:
a processing unit operable to: obtain a dataset of immune marker expression profiles in a leukocyte sample taken from the patient with cancer; sort the dataset based on a probability that an immune marker expression of a plurality of cells in the leukocyte sample will correspond to patients with cancer that respond to selective internal radiation therapy (SIRT) or not respond to or only transiently respond to SIRT; and allocate a probability score to the sample taken from the patient with cancer that the patient will (i) respond to SIRT or (ii) not respond or only transiently respond to SIRT treatment, wherein the probability score of the sample is obtained from the majority of the plurality of cells having the immune marker expression corresponding to patients with cancer that respond to SIRT or corresponding to patients with cancer that do not respond or transiently respond to SIRT.

Another aspect of the invention provides use of a composition comprising a SIRT and an immunotherapy in the manufacture of a medicament for use in the treatment of cancer in a patient prognosticated as a sustained responders (SR) to selective internal radiation therapy (SIRT), preferably hepatocellular carcinoma.

Another aspect of the invention provides a method of treating a patient with cancer that is prognosticated as a sustained responders (SR) to selective internal radiation therapy (SIRT) comprising administering a therapeutically effective dose of a composition comprising a SIRT and an immunotherapy to the patient.

Another aspect of the invention provides a composition comprising a selective internal radiation therapy (SIRT) and an immunotherapy for use in the treatment of cancer in a patient prognosticated as a sustained responder (SR) to SIRT.

Other aspects of the invention will become apparent to those of ordinary skill in the art upon review of the following description of specific embodiments of the invention in conjunction with the accompanying figures.

### BRIEF DESCRIPTION OF THE DRAWINGS

The present invention will now be described, by way of example only, with reference to the following accompanying drawings.

FIG. 1: Flow of model for prediction of sustained response in HCC patients after Y90 radioembolization. Single cell CyTOF data with 37 markers were used to obtain a final prediction class status for each individual patient as a responder or non-responder.

FIG. 2: Prediction model for sustained response to Y90-RE using random forest prediction method. A. Dendrogram showing distance between samples in the training cohort n=22 and testing cohort n=8 based on the random forest prediction model. B. Accuracy curve based on mtry (number of random variables) and ntree (number of trees) for the training model using random forest prediction method. The most accurate parameters selected was mtry=10 and ntree=2000 with an accuracy of 76.8% C. A depiction of how the clinical outcomes are predicted D. Receiver operating characteristic (ROC) curve from Random Forests prediction method to predict sustained response after Y90-RE in the training cohort n=22 and validation/testing cohort n=8. AUC=area under the curve.

FIG. 3: ROC curve of training set model and testing set model at single cell level, using the final predictive model from random forest prediction modeling. Training set n=22: AUC=0.8461 and accuracy=0.7686; Testing set n=8: AUC=0.6769 and accuracy=0.7082 AUC=area under the curve.

FIG. 4: Prediction model for sustained response to Y90-RE showing a series of immune responses induced by Y90-RE in tumor infiltrating lymphocytes (TILs) and PBMCs. In TILs from Y90-RE treated tumors (resections were performed between 3 to 6 months after Y90-RE upon successful downstaging), an increase in infiltration of GB-expressing CD8+, natural killer (NK) cells and NKT cells was observed after Y90-RE versus more TREG in the treatment-naïve control tumors. Y90-RE-induced upregulation of chemokines within tumor microenvironment is hypothesized to link to CD8+ T cells recruitment and activation. In PBMCs on the other hand, at 1 month (1 mo) and 3 months (3 mo) post-Y90-RE, immune activation of TNFα-expressing and granzyme B (GB)-expressing immune subsets and antigen-presenting cells (APCs) was observed.

FIG. 5: Immune profiles of tumor infiltrating leukocytes (TILs) isolated from Y90-RE-treated and treatment-naive tumors A. Samples collection and analysis pipeline. PBMCs were collected before (Pre Y90) and at various time points after Y90-RE (Post Y90) (n=31 patients). TILs were collected from resected HCC tumors from post Y90-RE (downstaged upon therapy) or treatment-naive patients, Ctl (n=7 for each group). CyTOF was used to analyze both the PBMCs and TILs and NGS was performed on tumor tissues from post-Y90-RE and treatment naive patients (n=4 for each group). B. 2D heat map showing the differential expression of immune markers by nodes enriched in TILs isolated from post Y90-RE (upper left bar) or treatment naive (Ctl; upper right bar) HCC tumors. Enriched immune subsets in TILs from post Y90-RE were CD56+ natural killer (NK) cells, CD8+CD56+ NKT cells, CD8+ Tim3+ T and CD4+CD45RO+ T cells while regulatory T, Treg cells were enriched in TILs from Ctl HCC (grey scale-coded lines). n=7 each group.

FIG. 6. 2D representation of A granzyme B (GB) and B Tim-3 expression on TILs isolated from post-Y90-RE (Y90) and Ctl HCC tumors. Images were generated using inhouse enhanced ACCENSE software.

FIG. 7. Representative plots showing the gating of GB on CD8+ T cells from post-Y90-RE (Y90) or Ctl TILs (Top panel). Percentage of GB+CD8+ and Tim-3+CD8+ T cells from post-Y90-RE and Ctl TILs (bottom panel).

FIG. 8. Percentage of CD56+NK cells, CD8+CD56+ NKT cells, GB+CD56+NK cells and GB+CD8+CD56+ NKT cells from post-Y90-RE (Y90) and Ctl TILs. Graphical data represent the means±standard deviations and were analyzed by unpaired Student's t-test. * P<0.05 and ** P<0.01.

FIG. 9: Immune profiles of TILs from post-Y90 RE or treatment naive tumors. A density plots showing CXCR3 and CD45RO expression from representative nodes, NODE 244 representing CD4+ population that was enriched in TILs from post Y90 RE tumors. Grey plots show expression profiles from the selected node and black plots show expression profiles of all the nodes. Grey and black lines represent the average expression level of each marker. B graphs show percentages of CXCR3+CD4+CD45RO+ T cells in TILs from post Y90 RE (Y90) and treatment naïve (Ctl) tumors. Graphs represent the means±SDs and were analyzed by unpaired Student's t-test. * p<0.05.

FIG. 10: Immune profiles of TILs from post-Y90 RE or treatment naive tumors. Representative plots showing the gating of Foxp3+CD152+ Treg cells on pre-gated CD4+ T cells from post Y90 RE (Y90) and treatment naive (Ctl) TILs. Right, graphs shows percentage of Treg cells. Data show means±SDs and were analyzed by unpaired Student's t-test. * p<0.05.

FIG. 11: Chemotatic pathways involving CCL5 and CXCL16 induced by Y90-RE. A. RNA expression of CCL5 and CXCL16 in Y90-treated (n=8) versus Ctl (n=6) tumor tissues by quantitative PCR analysis. B Correlation between RNA expression of CCL5 and CXCL 16 and the percentage of tumor-infiltrating GB+CD8+-activated T cells (n=14). Graphical data represent the means±standard deviations. P values and correlation coefficients (r) were calculated using the Pearson's correlation test. * P<0.05 and ** P<0.01.

FIG. 12: Immune profiles of PBMCs from responders versus non responders to Y90 RE linked to clinical response detected in peripheral blood mononuclear cells (PBMCs) at 1 month and 3 months post-Y90-RE. A Representative plots showing the gating of CD8+ Tim3+ T cells (upper panels) and TNF-α expression gated on these CD8+ Tim3+ T cells (lower panels) from PBMCs isolated before (Pre) and 1-month (1 mo) after Y90-RE. Right panel shows the percentage of TNF-α expressing CD8+ Tim-3+ T cells in sustained-responders (SRs) or non-responders (NRs) and transient-responders (TRs) to Y90-RE before (Pre) and after (1-6 mo) therapy. B. Representative plots showing the gating on TNF-α expressing CD4+ T cells from PBMCs isolated before (Pre) or 1-month (1 mo) after Y90 therapy. Right panels show the percentage of TNF-α expressing CD4+ T cells. C. Representative plots showing the gating on CD14+HLA-DR+ cells from PBMCs isolated before (Pre) and 3-months (3 mo) after Y90-RE. Right panel shows the percentage of CD14+HLADR+ antigen-presenting cells in SRs and NRs/TRs to Y90-RE before (Pre) and after (1 mo-6 mo) therapy. Graphical data represent the means±standard deviation. Data were analyzed by paired Student's t-test (for pre versus 1 mo or 3 mo) or unpaired Student's t-test (for 3 mo SRs versus NRs/TRs). * P<0.05. ** P<0.01. D. Density plots showing CD14 and HLA-DR expression from representative nodes, NODE 293 represents APCs enriched in TILs from post Y90 RE tumors. Red plot shows expression profile from the selected node and black shows that of all the nodes. Red and black lines represent the average expression levels of each marker. E. Representative plots show the gating of GB+CD56+NK cells from pre- and 3 mo post Y90 RE. Right, graphs show percentages of GB+CD56+NK cells pre and at various time points post-Y90 RE in sustained-responders (SRs) and non- or transient responders (NRs/TRs). Data shows means±SDs and were analyzed by unpaired Student's t-test. * p<0.05.

FIG. 13: Distinct immune subsets in sustained-responders (SRs) versus non-responders (NRs)/transient-responders (TRs) to Y90-RE. A Graphs show percentages of CXCR6+ or CCR5+CD8+ T cells and CXCR6+ or CCR5+CD8+ Tim-3 T cells in PBMCs isolated from SRs or NRs/TRs patients before (Pre) and at various time points (1, 3 and 6 mo) after Y90-RE. Graphical data represent the means±standard deviation and were analyzed by unpaired Student's t-test. * P<0.05 and ** P<0.01. B 2D cellular illustration of PD-1 and Tim-3 on PBMCs isolated before (Pre) and at 3-months (3 mo) after Y90-RE from SRs or NRs/TRs patients. Images were generated using ACCENSE software.

FIG. 14: Distinct immune subsets in sustained-responders (SRs) versus non-responders (NRs)/transient-responders (TRs) to Y90-RE. A Graphs show the percentages of the immune subsets: PD-1+CD8+ T cells, Tim3+CD8+ T cells, PD-1+CD45RO+CD4+ T cells and Foxp3+CD152+ Treg from SRs and NRs/TRs at various time points (1, 3 and 6 mo) after Y90-RE. B Representative plots showing percentages of PD-1+CD8+ T cells and Tim3+CD8+ T cells C. Representative plots showing percentages of PD1+CD4+ T cells (pre gated on CD4+CD45RO+ T cells), D Representative plots showing percentages of Foxp3+CD152+ Treg cells (pre gated on CD4+ T cells) B-D Immune subsets from pre-Y90 RE PBMCs in sustained responders (SRs) versus non or transient responders (NRs/TRs).

FIG. 15: Distinct immune subsets in sustained-responders (SRs) versus non-responders (NRs)/transient-responders (TRs) to Y90-RE. A 2D cellular illustration of CXCR6 and CCR5 on PBMCs isolated before (Pre) and 3-months (3 mo) after Y90-RE from SRs and NRs/TRs patients. Images were generated using ACCENSE software. B Representative plots showing percentages of CCR5+ or CXCR6+CD8+ T cells. Immune subsets from pre-Y90 RE PBMCs in sustained responders (SRs) versus non- or transient responders (NRs/TRs).

### DETAILED DESCRIPTION OF THE INVENTION

Unless defined otherwise, all technical and scientific terms used herein have the same meaning as is commonly understood by a skilled person to which the subject matter herein belongs. As used herein, the following definitions are supplied in order to facilitate the understanding of the present invention.

Throughout this document, unless otherwise indicated to the contrary, the terms “comprising”, “consisting of”, “having” and the like, are to be construed as non-exhaustive, or in other words, as meaning “including, but not limited to”.

Furthermore, throughout the specification, unless the contest requires otherwise, the word “include” or variations such as “includes” or “including” will be understood to imply the inclusion of a stated integer or group of integers but not the exclusion of any other integer or group of integers.

As used in the specification and the appended claims, the singular form “a”, and “the” include plural references unless the context clearly dictates otherwise.

The immune profile of surgically resected HCC, which has been downstaged by Y90-RE were analysed. Using time-of-flight mass-cytometry (CyTOF) for high-dimensional, in-depth immunophenotyping key anti-tumour immune responses induced by the Y90-RE have been identified that may underlie the clinical response. Next-generation sequencing (NGS) of tumour tissues from patients after Y90-RE identified activation of multiple immune-subsets and a potential pathway that induces the recruitment of activated CD8+ T cells. The immune profiles of the peripheral-blood-mononuclear-cells (PBMCs) from patients before and at various time points after Y90-RE were also examined and key immune-subsets were identified, including CD8+ T cells and CD4+ T cells expressing the checkpoint receptors PD-1 and Tim-3 and homing receptors CCR5 and CXCR6 in those who responded to Y90-RE. In addition, a prediction model was built using the immune profile of pre-therapy PBMCs to identify potential sustained responders to Y90-RE.

Accordingly, an aspect of the invention provides an in vitro method for the prognosis of response to treatment for a patient suffering from cancer, the method comprising: measuring expression of at least one immune marker in a leukocyte sample taken from patient with cancer; classifying the patient sample into (i) sustained responder (SR) to selective internal radiation therapy (SIRT) or (ii) transient/non-responders (TR/NR) to SIRT based on the expression of the at least one immune marker in relation to a predetermined value.

As used herein the term ‘prognoses”, ‘prognosis’, ‘prognosed’ ‘prognosticate’, ‘prognosticated’, or ‘prognosing’ relates to providing a forecast or prediction of the likely outcome of a cancer after treatment with selective internal radiation therapy (SIRT). SIRT is a form of radiation therapy used in interventional radiology to treat cancer. It is generally for selected patients with surgically unresectable cancers. The treatment involves injecting tiny glass or resin microspheres containing radioactive material preferably yttrium 90 isotope (Y-90) into the arteries that supply the tumour. Because this treatment combines radiotherapy with embolization, it is also called radioembolization. The forecast or prediction of the likely outcome of a cancer after treatment with selective internal radiation therapy (SIRT) may be the predicted outcome of either a sustainable responder (SR) to selective internal radiation therapy (SIRT) or transient/non-responders (TR/NR) to SIRT over a term of 1 month, or 3 months, or 6 months or 12 months or 24 months In various embodiments the forecast or prediction of the likely outcome of a cancer after treatment with selective internal radiation therapy (SIRT) may be based on Response Evaluation Criteria in Solid Tumours (RECIST) 1.1 guidelines (Eisenhauer, et al. Eur J Cancer 45, 228-247 (2009).) wherein sustained responders (SRs) were defined as patients with a likely outcome of no progressive disease (non-PD) by 6 months (180 days) after Y90 RE; while the non-responders (NRs) were defined as patients with a likely outcome of progressive disease (PD) even at 3 months; or transient-responders (TRs) with a likely outcome of a an initial response (non-PD) at 3 months but progressed (PD) by 6 months after Y90 RE.

As used herein a patient suffering from cancer refers to a subject diagnosed with an unresectable cancer, and/or a hyper-vascularized cancer. In various embodiments the cancer may be one of the following unresectable and/or hyper-vascularized cancers: hepatic cell carcinoma (HCC); liver cancer; colorectal cancer; neuroendocrine tumour; HCC patients who are currently ineligible for liver transplant; metastatic liver cancer; metastatic colorectal cancer; metastatic neuroendocrine tumour; a Cancer where a hyper-vascularized mass has developed around the cancer or any other cancer wherein the patient has a life expectancy of at least 3 months.

As used herein the term ‘classifying’, ‘classified’, or ‘classification’, refers to separating a patient population into subpopulations according to specified criteria or a process of determining or arranging patients into a particular group depending on the immune marker profile of a sample taken from the patient or a process or sorting a patient as having a particular prognosis such as a sustainable responder (SR) to selective internal radiation therapy (SIRT) or transient/non-responders (TR/NR) to SIRT. In various embodiments the patient may be classified for different treatment protocols such as treatment with SIRT alone or treatment with SIRT in combination with immunotherapy whereby patients classified as a sustainable responder (SR) to selective internal radiation therapy (SIRT) will be treated with SIRT alone or SIRT in combination with immunotherapy.

In various embodiments the expression of at least one immune marker is measured using mass spectrometry by time of flight (CyTOF) or Next-generation sequencing (NGS), or quantitative polymerase chain reaction (qPCR) or any other method known in the art to measure expression levels of an immune marker.

In various embodiments at least one immune marker is selected from PD-1 (SEQ ID NO. 9), Tim-3 (SEQ ID NO. 21), CXCR6 (SEQ ID NO. 12), or combinations thereof, co-expression of PD-1 (SEQ ID NO. 9) or Tim-3 (SEQ ID NO. 21) with CCR5 (SEQ ID NO. 34). In various embodiments the at least one immune marker comprises PD-1 (SEQ ID NO. 9). In various embodiments the at least one immune marker comprises Tim-3 (SEQ ID NO. 21). In various embodiments the at least one immune marker comprises CXCR6 (SEQ ID NO. 12). In various embodiments the at least one immune marker comprises co-expression of PD-1 (SEQ ID NO. 9) with CCR5 (SEQ ID NO. 34). In various embodiments the at least one immune marker comprises co-expression of Tim-3 (SEQ ID NO. 21) with CCR5 (SEQ ID NO. 34). In various embodiments the at least one immune marker comprises co-expression of PD-1 (SEQ ID NO. 9) with CXCR6 (SEQ ID NO. 12). In various embodiments the at least one immune marker comprises co-expression of Tim-3 (SEQ ID NO. 21) with CXCR6 (SEQ ID NO. 12). In various embodiments the at least one immune marker is any one of PD-1 (SEQ ID NO. 9), Tim-3 (SEQ ID NO. 21), CCR5 (SEQ ID NO. 34) or CXCR6 (SEQ ID NO. 12) co-expression on a CD8+ T-cell or a CD4 T-cell.

In various embodiments prior to measuring expression of the at least one immune marker a prediction model is created comprising:
Measuring expression of a plurality of immune markers in a sample taken from a plurality of patients with cancer;
Evaluating a clinical response of the patients with cancer to treatment with (SIRT) at 3 and/or 6 months after treatment wherein the clinical response is selected from (SR), and (TR/NR);
Determining if there is a correlation between each immune marker and either the clinical response (SR), or the clinical response (TR/NR);
determining the probability of each immune marker one by one to positively affect the accuracy of the clinical response;
allocating a probability score to each sample based on the number of immune markers that positively affects the accuracy of the clinical response; and
allocating the same probability score for each immune marker present in a leukocyte sample taken from a new patient with cancer to classify the new patient into predicted SR or predicted TR/NR.

In various embodiments the plurality of immune markers are any combination of two or more immune markers listed in table 3, including CD14 (SEQ ID NO. 1); CD3 (SEQ ID NO. 2); CD19 (SEQ ID NO. 3); CD45RO (SEQ ID NO. 4); HLA-DR (SEQ ID NO. 5); CD8 (SEQ ID NO. 6); T-bet (SEQ ID NO. 7); CD28 (SEQ ID NO. 8); PD-1 (SEQ ID NO. 9); CD154 (SEQ ID NO. 10); CD103 (SEQ ID NO. 11); CXCR6 (SEQ ID NO. 12); TNF-α (SEQ ID NO. 13), CD25 (SEQ ID NO. 14); CD27 (SEQ ID NO. 15); CD152 (SEQ ID NO. 16); PD-L1 (SEQ ID NO. 17); CD244 (SEQ ID NO. 18); IL-10 (SEQ ID NO. 19); LAG-3 (SEQ ID NO. 20); TIM-3 (SEQ ID NO. 21); CCR7 (SEQ ID NO. 22); CD56 (SEQ ID NO. 23); CXCR3 (SEQ ID NO. 24); GITR (SEQ ID NO. 25); FoxP3 (SEQ ID NO. 26); KI67 (SEQ ID NO. 27); CD80 (SEQ ID NO. 28); IFN-γ (SEQ ID NO. 29); IL-17A (SEQ ID NO. 30); EOMES (SEQ ID NO. 31); Granzyme B (SEQ ID NO. 32); CD37 (SEQ ID NO. 33); CCR5 (SEQ ID NO. 34); CD4 (SEQ ID NO. 42); or CD69 (SEQ ID NO. 35). In various embodiments the plurality of immune markers are any combination of three or more immune markers listed in table 3; or four or more immune markers listed in table 3; or five or more immune markers listed in table 3; or six or more immune markers listed in table 3; or seven or more immune markers listed in table 3; or eight or more immune markers listed in table 3; or nine or more immune markers listed in table 3; or ten or more immune markers listed in table 3; or eleven or more immune markers listed in table 3; or twelve or more immune markers listed in table 3; or thirteen or more immune markers listed in table 3; or fourteen or more immune markers listed in table 3; or fifteen or more immune markers listed in table 3; or sixteen or more immune markers listed in table 3 including CD14; CD3; CD19; CD45RO; HLA-DR; CD8; T-bet; CD28; PD-1; CD4; CD154; CD103; CXCR6; CD25; CD27; CD152; PD-L1; CD244; IL-10, LAG-3; TIM-3; CCR7; CD56; CXCR3; GITR; FoxP3; KI67; CD80; IL-17A; EOMES; Granzyme B; CD37; CCR5; or CD69. The expression of a plurality of immune markers in a sample taken from a plurality of patients with cancer are taken before the patients with cancer are treated with SIRS.

In various embodiments the plurality of patients with cancer may include 3 to 1000 patients, 3 to 500 patients; 3 to 200 patients; 3 to 100 patients; 3 to 90 patients; 3 to 80 patients; 3 to 70 patients; 3 to 60 patients; 3 to 50 patients; 3 to 40 patients; 3 to 30 patients; 3 to 20 patients; 3 to 10 patients, or any number of patients more than 2 that is suitable to create a prediction model that is at least as accurate as the classification of the patient samples based on the expression of the at least one immune marker in relation to the predetermined value to predict whether the patient will respond to treatment with (SIRT) mentioned above.

In various embodiments the evaluation of the clinical response of the patients with cancer to treatment with (SIRT) is based on the Response Evaluation Criteria in Solid Tumours (RECIST) 1.1 guidelines (Eisenhauer, et al. Eur J Cancer 45, 228-247 (2009).) wherein sustained responders (SRs) were defined as patients with a likely outcome of no progressive disease (non-PD) by 6 months (180 days) after Y90 RE; while the non-responders (NRs) were defined as patients with a likely outcome of a minimal response of stable disease (SD) even at 3 months or transient-responders (TRs) with a likely outcome of a an initial response at 3 months but progressed by 6 months after Y90 RE.

In various embodiments the determination of a correlation between each immune marker and either the clinical response (SR), or the clinical response (TR/NR); the determination of the probability of each immune marker to positively affect the accuracy of the clinical response and the allocation of a probability score to each sample based on the number of immune markers that positively affects the accuracy of the clinical response is accomplished using a machine leaning algorithm such as a random forest prediction model random forest. Other similar weighted neighbourhood schemes may also be used. In various embodiments the random forest comprises models built from a training set mathematically expressed as:—

$${\{ {x}_{i} , {y}_{i} \}}^{n} i = 1 \qquad ( 1 )$$

Wherein x refers to an immune marker, y refers to a clinical response, i refers to a number of cells and n refers to the number of trees.
This makes predictions ŷ for new points x′ by looking at the “neighbourhood” of the point, formalized by a weight function W mathematically expressed as:

$$\hat{y} = {\sum }_{i = 1}^{n} W ( {x}_{i} , {x}^{′} ) {y}_{i}$$

Here, W (xᵢ, x′) is the non-negative weight of the i'th training point relative to the new point x′ in the same tree. For any particular x′, the weights for points xᵢ, must sum to one. Weight functions are given as follows:
In k-NN, the weights are

$$W ( {x}_{i} , {x}^{′} ) = \frac{1}{k}$$

if xᵢ is one of the k points closest to x′, and zero otherwise.
In a tree,

$$W ( {x}_{i} , {x}^{′} ) = \frac{1}{k} ,$$

if xᵢ is one of the k′ points in the same leaf as x′, and zero otherwise.

Since a forest averages the predictions of a set of m trees with individual weight functions Wⱼ, its predictions are

$$\hat{y} = \frac{1}{m} {\sum }_{j + 1}^{m} {\sum }_{i = 1}^{n} {W}_{j} ( {x}_{i} , {x}^{′} ) {y}_{i} = {\sum }_{i + 1}^{n} ( \frac{1}{m} {\sum }_{j + 1}^{m} {W}_{j} ( {x}_{i} , {x}^{′} ) {y}_{i}$$

This shows that the whole forest is again a weighted neighbourhood scheme, with weights that average those of the individual trees. The neighbours of x′ in this interpretation are the points Xᵢ sharing the same leaf in any tree j. In this way, the neighbourhood of x′ depends in a complex way on the structure of the trees, and thus on the structure of the training set. The shape of the neighbourhood used by a random forest adapts to the local importance of each feature wherein m refers to the number of random variables. In various embodiments n is 10 and m is 2000.

In various embodiments the determination of the probability of each immune marker to positively affect the accuracy of the clinical response is at a cellular level that determines the probability that an immune marker expressed in relation to an individual cell will have a sustained response to SIRT or not. In various embodiments single-cell expression data of about 10,000 single cells with a plurality of immune markers from each patient from randomly selected cancer patients may be used. In various embodiments single-cell expression data is downsampled to about 10,000 single cells to improve the accuracy of the single cell level.

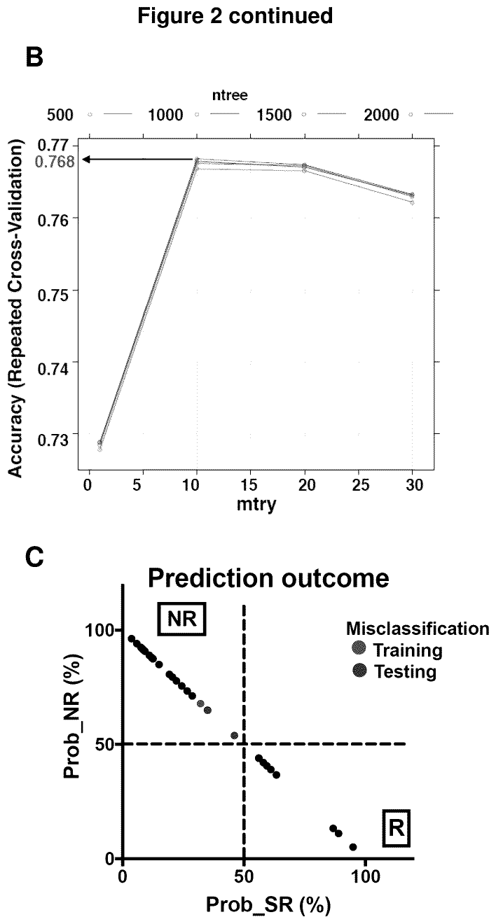

In various embodiments the allocation of a probability score to each sample based on the number of immune markers that positively affects the accuracy of the clinical response is at a sample level that compares the neighbouring cell expression profiles in each sample and allocates the probability score to each sample based on the number of immune markers that positively affects the accuracy of the clinical response. In various embodiments for each of the samples, a percentage of cells being classified as SR or NR is computed and used as a voting system for probability scores, where ≥50% as SR classifies the samples in SR group while <50% as SR classifies them in NR group (FIG. 2C). This final step of voting system generates highly accurate prediction outcomes. The single cell training model and sample based voting system, provides highly accurate prediction of response to SIRT Y90 RE than any other clinical parameters.

In various embodiments the new patient or new patients then become a test set. The new patient may be one or more new patients or any number of new patients. In various embodiments the new patient has not been treated with SIRT when the sample is taken.

In various embodiments the method further comprises comparing the predicted SR or predicted TR/NR with the classification of the patient samples based on the expression of the at least one immune marker in relation to the predetermined value to predict whether the patient will respond to treatment with (SIRT).

The comparison of the testing set of new patients and the classification of the patient samples based on the expression of the at least one immune marker in relation to the predetermined value to predict whether the patient will respond to treatment with (SIRT) allows for checking the accuracy of the prediction to ensure the new patient receives the most appropriate treatment, whereby patients predicted to be a sustainable responder (SR) to selective internal radiation therapy (SIRT) will be treated with SIRT alone or with SIRT in combination with immunotherapy. Similarly, patients predicted to be transient/non responders (TR/NR) will not be treated with SIRT and would instead be treated with TACE, Sorafenib or immunotherapy alone.

In various embodiments the immune marker allocated with a higher probability to positively affect the accuracy of the clinical response comprises the at least one immune marker.

In various embodiments at least one immune marker is selected from PD-1, Tim-3, CXCR6, or combinations thereof, co-expression of PD-1 or Tim-3 with CCR5. In various embodiments the at least one immune marker comprises PD-1. In various embodiments the at least one immune marker comprises Tim-3. In various embodiments the at least one immune marker comprises CXCR6. In various embodiments the at least one immune marker comprises co-expression of PD-1 with CCR5. In various embodiments the at least one immune marker comprises co-expression of Tim-3 with CCR5. In various embodiments the at least one immune marker comprises co-expression of PD-1 with CXCR6. In various embodiments the at least one immune marker comprises co-expression of Tim-3 with CXCR6. In various embodiments the at least one immune marker is any one of PD-1, Tim-3, CCR5 or CXCR6 co-expression on a CD8+ T-cell.

In various embodiments the leukocyte sample comprises a tumour infiltrating leukocyte sample. In various other embodiments the leukocyte sample comprises a peripheral blood mononuclear cell (PBMC) sample. In various embodiments the leukocyte sample comprises a T-cell expressing CD 8. In various embodiments the leukocyte sample comprises a T-cell expressing CD 4. In various embodiments the sample is analysed using mass spectrometry by time of flight (CyTOF)

In various embodiments the cancer is hepatocellular carcinoma.

In various embodiments the leukocyte sample taken from patient with cancer, including a test patient with cancer predicted or classified as sustainable responder (SR) is identified as requiring treatment with SIRT alone or a combination of SIRT and an immunotherapy. SIRT and Immunotherapy like other therapies have side effects and add to the cost of any treatment. Therefore the advantage of first predicting if a patient with cancer will respond to SIRT is that only patients with cancer that will have a sustained response to the SIRT treatment will be given SIRT alone or SIRT with the additional immunotherapy. Similarly, patients predicted to be transient/non responders (TR/NR) will not be treated with SIRT and would instead be treated with TACE, Sorafenib or immunotherapy alone.

As used herein the term ‘immunotherapy’ may refer to active, passive or a hybrid of active and passive immunotherapy. Active immunotherapy directs the immune system to attack tumour cells and may involve the removal of immune cells from the blood or from a tumor. Those specific for the tumor are cultured and returned to the patient where they attack the tumor; alternatively, immune cells can be genetically engineered to express a tumor-specific receptor, cultured and returned to the patient. In various embodiments this may include Adoptive T-cell therapy. Passive immunotherapies enhance existing anti-tumor responses and include the use of monoclonal antibodies, lymphocytes or cytokines. In various embodiments the immunotherapy may comprise antibodies that inhibit tyrosine kinase; programmed cell death 1 receptor (PD-1); Hepatitis A virus cellular receptor 2 (HAVCR2), also known as T-cell immunoglobulin and mucin-domain containing-3 (Tim-3); vascular endothelial growth factor; (VEGF); epidermal growth factor receptor (EGFR); cytotoxic T-lymphocyte-associated protein 4 (CTLA-4) or Lymphocyte-activation gene 3 (Lag-3). Suitable antibodies may be selected from sunitinib; erlotinib; ipilimumab; nivolumab; pembrolizamab and bevacizumab. In various embodiments the immunotherapy may comprise cytokines or chemokines for example interferons such as TNFα or interleukins such as IL-2. In various embodiments the immunotherapy may comprise combinations of any of the above listed immunotherapies.

Another aspect of the invention provides a system for prognosing a response to treatment for a patient suffering from cancer, the system comprising: a processing unit operable to: obtain a dataset of immune marker expression profiles in a leukocyte sample taken from the patient with cancer; sort the dataset based on a probability that an immune marker expression of a plurality of cells in the leukocyte sample will correspond to patients with cancer that respond to selective internal radiation therapy (SIRT) or not respond to or only transiently respond to SIRT; and allocate a probability score to the sample taken from the patient with cancer that the patient will (i) respond to SIRT or (ii) not respond or only transiently respond to SIRT treatment, wherein the probability score of the sample is obtained from the majority of the plurality of cells having the immune marker expression correspond to patients with cancer that respond to SIRT or correspond to patients with cancer that do not respond or transiently respond to SIRT.

The processing unit may be any known processing unit able to obtain, sort and allocate the dataset as described herein.

In various embodiments the system further comprising a device for measuring expression of immune markers in a leukocyte sample taken from the patient with cancer.

In various embodiments the processing unit forms part of the device for measuring expression of immune markers in a leukocyte sample taken from the patient with cancer.

In various embodiments the device is a cytometer. In various embodiments the cytometer is a mass cytometer suitable for combined plasma mass spectrometry and time of flight mass spectrometry. In this approach, antibodies are conjugated with isotopically pure elements, and these antibodies are used to label both ubiquitous and targeted immune markers such as leukocyte proteins. Cells are nebulized and sent through an argon plasma, which ionizes the metal-conjugated antibodies. The metal signals are then analyzed by a time-of-flight mass spectrometer. In various other embodiments the cytometer may be a flow cytometer that uses conjugated fluorophores rather than isotopes or any other cytometer known in the art to defect and/or measure expression of immune markers in a leukocyte.

In various embodiments the device is a next generation sequencer such as pyrosequencers, Hi-Seq genome sequencers (illumina), massively parallel signature sequencer, or any other high-throughput sequencing device or system known in the art to defect and/or measure mRNA expression of immune markers in a leukocyte. In various embodiments the device is a thermocycler. In various embodiments the thermocycler can be used for measuring quantitative Polymerase Chain Reaction (qPCR) or real time qPCR (RT qPCR). In various embodiments the device is any device or system known in the art to defect and/or measure mRNA expression of immune markers in a leukocyte.

Another aspect of the invention provides a composition comprising a selective internal radiation therapy (SIRT) and an immunotherapy for use in the treatment of cancer in a patient prognosticated as a sustained responder (SR) to SIRT.

A SIRT may comprise tiny glass or resin microspheres containing radioactive material. In various embodiments the SIRT is yttrium 90 isotope (Y-90). In various embodiments the immunotherapy may include Adoptive T-cell therapy. In various embodiments the immunotherapy may include T-cells engineered to be directed against tumour antigens. In various embodiments the immunotherapy may comprise antibodies that inhibit tyrosine kinase; programmed cell death 1 receptor (PD-1); Hepatitis A virus cellular receptor 2 (HAVCR2), also known as T-cell immunoglobulin and mucin-domain containing-3 (Tim-3); vascular endothelial growth factor; (VEGF); epidermal growth factor receptor (EGFR); cytotoxic T-lymphocyte-associated protein 4 (CTLA-4) or Lymphocyte-activation gene 3 (Lag-3). Suitable antibodies may be selected from sunitinib; erlotinib; ipilimumab; nivolumab; pembrolizamab and bevacizumab. In various embodiments the immunotherapy may comprise cytokines or chemokines for example interferons such as TNFα or interleukins such as IL-2. In various embodiments the immunotherapy may comprise combinations of any of the above listed immunotherapies.

In various embodiments the immunotherapy may be included on the microspheres together with the yttrium 90 isotope (Y-90). In various other embodiments the immunotherapy may be provided separately from the microspheres with the yttrium 90 isotope (Y-90).

In various embodiments the composition is suitable for use in the treatment of cancer. In various embodiments the cancer is hepatocellular carcinoma.

Another aspect of the invention provides use of a composition comprising a selective internal radiation therapy (SIRT) and an immunotherapy in the manufacture of a medicament for use in the treatment of cancer in a patient prognosticated as a sustained responder (SR) to SIRT, preferably hepatocellular carcinoma. Wherein the composition comprising a SIRT and an immunotherapy is as described above herein.

Another aspect of the invention provides a method of treating a patient with cancer that is prognosed as a sustained responder (SR) to selective internal radiation therapy (SIRT) comprising administering a therapeutically effective dose of a composition comprising a SIRT or a SIRT and an immunotherapy to the patient. Wherein the composition comprising a SIRT and an immunotherapy is as described above herein. Similarly, a method of treating a patient with cancer that is prognosed as a transient/non-responder (TR/NR) to selective internal radiation therapy (SIRT) comprises administering other therapies rather than SIRT such as TACE or Sorafenib or immunotherapy to the patient.

In various embodiments the patient with cancer comprises a patient with hepatocellular carcinoma.

In various embodiments the immunotherapy may be administered together with the SIRT.

In various other embodiments the immunotherapy may be administered separately from the SIRT.

### EXAMPLES

Time-of-flight mass cytometry and next-generation sequencing (NGS) were used to analyze the immune landscapes of tumor-infiltrating leukocytes (TILs), tumor tissues and peripheral blood mononuclear cells (PBMCs) at different time-points before and after Y90-RE.

TILs isolated after Y90-RE exhibited markers indicative of local immune activation, including high expression of granzyme B (GB) and infiltration of CD8+ T cells, CD56+NK cells and CD8+CD56+ NKT cells. NGS confirmed the upregulation of genes involved in innate and adaptive immune activation in Y90-RE-treated tumors. Chemotactic pathways involving CCL5 and CXCL16 correlated with the recruitment of activated GB+CD8+ T cells to the Y90-RE-treated tumors. When comparing PBMCs before and after Y90-RE, an increase in TNFα was observed on both the CD8+ and CD4+ T cells as well as an increase in percentage of antigen presenting cells after Y90-RE, implying a systemic immune activation. A high percentage of PD-1+/Tim-3+CD8+ T cells co-expressing the homing receptors CCR5 and CXCR6 denoted Y90-RE responders. A prediction model was also built to identify sustained responders to Y90-RE based on the immune profiles from pre-treatment PBMCs.

High-dimensional analysis of tumor and systemic immune landscapes identified local and systemic immune activation that may explain the sustained response to Y90-RE. Potential biomarkers associated with a positive clinical response were identified and a prediction model was built to identify sustained responders prior to treatment.

### Patients and Sample Processing

Tumour tissues and blood samples were obtained from a total of 41 patients with HCC from the National Cancer Center Singapore and Singapore General Hospital who were treated with or without prior Y90 RE therapy. Among which, n=14 underwent surgical resection for HCC and tumor-Infiltrating leukocytes (TILs) were isolated from the resected HCC tissue of 14 patients (Table 1) by enzymatic digestion.

TABLE 1
Clinical information for resected HCC from Y90 RE versus treatment naive patients, n = 12

| | Post Y90 | | | Treatment | Viral | TNM | | | | | |
|---|---|---|---|---|---|---|---|---|---|---|---|
| No. | RE | Resection* | No. | Naive | Status | Stage | Grade | Sex | CyTOF | NGS | qPCR |
| 1 | HEP157 | 190 | 8 | HEP174 | Hep B | 1 | II | M | + | + | + |
| 2 | HEP165 | 148 | 9 | HEP261 | Hep C | 2 | II | M | + | | + |
| 3 | HEP210 | 232 | 10 | HEP300 | Hep B | 1 | III | M | + | + | + |
| 4 | HEP242 | 182 | 11 | HEP304 | Hep B | 1 | II | F | + | + | + |
| 5 | HEP279 | 89 | 12 | HEP303 | Hep B | 3a | III | M | + | + | +* |
| 6 | HEP279 | 300 | 13 | HEP200 | NIL | 1 | III | M | + | | +* |
| 7 | HEP286 | 261 | 14 | HEP125 | Hep B | 1 | II | M | + | | |
| | Average | 202 | | | | | | | | | |

FootNote:
n = 7 Post Y90 RE versus n = 7 treatment-naive HCC tumors as controls (Ctl) with matched viral status, TNM Stage, Grade and Sex.
*number of days post Y90 RE when resection was performed
+ indicated the pair of samples taken for either CyTOF, NGS or qPCR experiments.
*Two tumor specimens were collected and analysed from HEP279 and HEP285 Y90 RE-treated tumours.

Peripheral blood mononuclear cells (PBMCs) were isolated from blood taken before (pre) and at various time points (1, 3 and 6 months) after Y90 RE from another cohort of 31 patients (Table 2 included 4 HCC patients who were subsequently resected after Y90 RE and their TILs were also analysed and included in Table 1) using conventional Ficoll (GE Healthcare, UK) isolation methods according to manufacturer's instructions.

TABLE 2
Clinical and samples collection information for HCC patients treated with Y90 RE

| | | | | | | 3 m | 6 m | Tumor | Location of | Response |
|---|---|---|---|---|---|---|---|---|---|---|
| No. | Pre | 1 mo | 3 mo | 6 mo | TIL | RECIST | RECIST | multiplicity | progression | status |
| 1 | + | + | | | | PD | PD | Multifocal | Non target | NR |
| | | | | | | | | | (distant) | |
| 2 | + | + | | | | PD | PD | 1 | Non target | NR |
| | | | | | | | | | (distant) | |
| 3 | + | | + | + | | SD | PD | Multifocal | Target | TR |
| 4 | + | | + | | | PD | PD | Multifocal | Non target | NR |
| | | | | | | | | | (distant) | |
| 5 | + | | + | + | | PD | PD | Multifocal | Non target | NR |
| | | | | | | | | | (distant) | |
| 6 | + | + | | | | PD | PD | 1 | Non target | NR |
| | | | | | | | | | (distant) | |
| 7 | + | + | + | + | | SD | PD | Multifocal | Non target | TR |
| | | | | | | | | | (liver) | |
| 8 | + | + | + | | | PD | PD | 1 | Target | NR |
| 9 | + | + | + | | | SD | PD | Multifocal | Non target | TR |
| | | | | | | | | | (distant) | |
| 10 | + | | + | | + | SD | PD | Multifocal | Non target | TR |
| | | | | | | | | | (liver) | |
| 11 | + | | | | | PD | PD | Multifocal | Target | NR |
| 12 | + | | | | | PD | PD | Multifocal | Non target | NR |
| | | | | | | | | | (distant) | |
| 13 | + | + | | | | PD | PD | Multifocal | Non target | NR |
| | | | | | | | | | (distant) | |
| 14 | + | + | | | | PD | PD | Multifocal | Non target | NR |
| | | | | | | | | | (distant) | |
| 15 | + | + | + | + | | PD | PD | Multifocal | Non target | NR |
| | | | | | | | | | (distant) | |
| 16 | + | | | | | PD | PD | Multifocal | Non target | NR |
| | | | | | | | | | (distant) | |
| 29 | + | | | + | | SD | PD | 1 | — | TR |
| 17 | + | | + | | | PD | PD | Multifocal | Non target | NR |
| | | | | | | | | | (distant) | |
| 18 | + | + | | | | PR | PR | 1 | — | SR |
| 19 | + | | + | + | | PR | PR | 1 | — | SR |
| 20 | + | + | + | | | SD | SD | Multifocal | — | SR |
| 21 | + | + | | + | | SD | SD | 1 | — | SR |
| 22 | + | + | + | + | | PR | PR | 1 | — | SR |
| 23 | + | + | | | | PR | PR | Multifocal | — | SR |
| 24 | | + | | | | SD | SD | 1 | — | SR |
| 25 | + | | + | + | + | PR | PR | Multifocal | — | SR |
| 26 | + | | + | + | + | PR | PR | 1 | — | SR |
| 27 | + | | + | | | SD | SD | 1 | — | SR |
| 28 | + | | + | | + | PR | PR | 1 | — | SR |
| 30 | + | + | + | | | PR | PR | 1 | — | SR |
| 31 | + | + | | | | SD | SD | Multifocal | — | SR |

Footnote:
+ Samples used in CyTOF
1 mo, 37 to 53 days; 3 mo, 75-146 days and 6 mo, 157 to 233 days
RECIST 1.1 criteria: CR, complete response is characterised by “disappearance of all target lesions”, PR, partial response is defined as “30% decrease in the sum of diameters of target lesions,” PD, progressive disease is defined as “20% increase in the sum of diameters of target lesions” and SD, stable disease is described as “neither sufficient shrinkage to qualify for PR nor sufficient increase to qualify for PD” (24).
Location of progression: target means progression at lesion treated with Y90; non-target means progression at lesions not treated with Y90 either within the liver (Liver); for lesions outside of liver such as lungs (Distant).
Response status: NR = non-responders, patients who are already PD at 3 m; TR = transient-responders, patients who are SD, PR or CR (non-PD) at 3 m but PD at 6 m; and SR = sustained responders, patients who are non-PD at 6 m.

Samples used for various analyses are denoted in Tables 1 and 2.

The Response Evaluation Criteria in Solid Tumors (RECIST) 1.1 guidelines (Eisenhauer, et al. Eur J Cancer 45, 228-247 (2009).) was used to evaluate tumour response. Sustained responders (SRs) were defined as patients without any progressive disease (non-PD) by 6 months (180 days) after Y90 RE; while the non-responders (NRs) never had a minimal response of stable disease (SD) even at 3 months; or transient-responders (TRs) who had an initial response at 3 months but progressed by 6 months after Y90 RE (Table 2). This study was approved by the Singapore Health Services-Central Institutional Review Board and all patients provided informed consent.

### Time-of-Flight Mass Cytometry (CyTOF)

TILs and PBMCs were analysed with 37 metal-conjugated antibodies (Table 3) using CyTOF as previously described (Chew et al. Proc Natl Acad Sci USA 114, E5900-E5909 (2017)). Briefly, immune cells were stained with cisplatin viability stain (DVS Sciences, USA) and anti-human CD45 leukocyte marker conjugated with lanthanide metal-89, 115 and 172 respectively—a triple-barcode system as previously described (Lai, et al. Cytometry A 87, 369-374 (2015)). The barcoded immune cells were combined and then stained with antibodies targeting surface markers. Cells were fixed with 1.6% paraformaldehyde and permeabilized in 100% methanol to permit intracellular antibody staining. Finally, a DNA intercalater (DVS Sciences, USA) was added for cellular visualization before analysis on a Helios mass cytometer (Fludigm, USA).

TABLE 3
Antibodies used for CyTOF staining

| Isotopes | Antibodies | Clone | Vendor |
|---|---|---|---|
| 89 | CD45 (Barcode 1) | HI30 | Fluidigm |
| 112/114 | CD14 | TüK4 | Life technologies |
| 115 | CD45 (Barcode 2) | HI30 | Biolegend |
| 139 | CD3 | UCHT1 | Biolegend |
| 141 | CD19 | HIB19 | Biolegend |
| 142 | CD45RO | UCHL1 | Biolegend |
| 143 | HLA-DR | L243 | Biolegend |
| 144 | CD8 | SK1 | Biolegend |
| 145 | T-bet | 4B10 | Biolegend |
| 146 | CD28 | CD28.2 | Biolegend |
| 147 | PD-1 | EH12.2H7 | Biolegend |
| 148 | CD4 | SK3 | Biolegend |
| 149 | CD154 | 24-31 | Biolegend |
| 150 | CD103 | B-Ly7 | Ebioscience |
| 151 | CXCR6 | K041E5 | Biolegend |
| 152 | TNF-α | Mab11 | Biolegend |
| 153 | CD25 | 2A3 | BD bioscience |
| 154 | CD27 | O323 | Biolegend |
| 155 | CD152 | BN13 | BD bioscience |
| 156 | PD-L1 | 29E.2A3 | Biolegend |
| 157 | CD244 | CL7 | Biolegend |
| 158 | IL-10 | JES3-9D7 | Biolegend |
| 159 | LAG-3 | 17B4 | Abcam |
| 160 | TIM-3 | F38-2E2 | Biolegend |
| 161 | CCR7 | G043H7 | Biolegend |
| 162 | CD56 | NCAM16.2 | BD bioscience |
| 163 | CXCR3 | G025H7 | Biolegend |
| 164 | GITR | 621 | Biolegend |
| 165 | FoxP3 | PCH101 | Ebioscience |
| 166 | Ki67 | 20Raj1 | Ebioscience |
| 167 | CD80 | 2D10 | Biolegend |
| 168 | INF-γ | B27 | Biolegend |
| 169 | IL-17A | BL168 | Biolegend |
| 170 | EOMES | 21Mags8 | Ebioscience |
| 171 | CD45RA | JS-83 | Ebioscience |
| 172 | CD45 (Barcode 3) | HI30 | Biolegend |
| 173 | Granzyme B | CLB-GB11 | Abcam |
| 174 | CD137 | 4B4-1 | Biolegend |
| 175 | CCR5 | T21/8 | Biolegend |
| 176 | CD69 | FN50 | Biolegend |
| 191/193 | Ir intercalator | | Fluidigm |

The Helios-generated output files were normalized using EQTM Four Element Calibration Beads (Cat #201078, Fluidigm) according to manufacturer's instructions (Finck, et al. Cytometry A 83, 483-494 (2013).) and de-barcoded manually by Boolean Gating strategy in FlowJo (version 10.2; FlowJo LLC, USA). Each sample was down-sampled to 10,000 live immune cells and equal number of samples were selected for each group before analysis using an in-house enhanced Automatic Classification of Cellular Expression by Nonlinear Stochastic Embedding (ACCENSE) software based on the combination of Barnes-Hut SNE non-linear dimension reduction algorithm and a k-means clustering algorithm (Shekhar, et al. Proc Natl Acad Sci USA 111, 202-207 (2014)). Cellular and nodal views of 2D t-Distributed Stochastic Neighbour Embedding (t-SNE) maps and density plots for the expression of individual markers in each node were generated simultaneously. Nodes that were significantly enriched (P<0.05) in either group were identified by paired or unpaired Mann-Whitney U test. All data were validated independently using FlowJo. Both 2D and 3D heat maps were plotted based on all significant nodes using R script for data visualization.

### Next-Generation Sequencing (NGS)

Tumour tissue from each patient was preserved in RNA Later (Thermo Fisher Scientific, USA) and stored at −80° C. until further processing. RNA was isolated using the mirVana miRNA Isolation Kit (Thermo Fisher Scientific) and cDNA was generated with the SMART-Seq® v4 Ultra™ Low Input RNA Kit for Sequencing

(Clontech, USA), according to manufacturers' protocols. Illumina-ready cDNA libraries were generated from amplified cDNA using the Nextera XT DNA Library

Prep Kit (Illumina, USA) and multiplexed for 2×101 bp-sequencing. NGS was performed externally at the Genome Institute of Singapore on a HiSeq High output platform.

Raw-sequencing reads were mapped via Hierarchical Indexing for Spliced Alignment of Transcripts (HISTAT) with reference to the Human Assembly GRCh38.p7. from Ensembl. Read alignments were then sorted using SAMtools and the raw gene counts were extracted with high-throughput sequencing data (HTSeq). The R package EdgeR tool was used for differential gene expression analysis between two sample groups. The empirical Bayes quasi-likelihood F-test was used in the Generalised Linear Model pipeline for gene-wise statistical analysis (Lund, et al. Stat Appl Genet Mol Biol 11, (2012)). Genes with a fold-change>2 and P<0.01 were selected. The data were then visualized in heat maps using R-Script and biological function analysis on enriched genes in post Y90 RE tumours was performed using the DAVID6.7 Functional Annotation Tool based on P<0.01 and benjamini<0.05 selection criteria. Additional functional pathway analysis of enriched genes was carried out using the Reactome Pathway Database (Croft, et al. Nucleic Acids Res 39, D691-697 (2011)).

Quantitative Polymerase Chain Reaction (qPCR)

RNA from tumour tissues was isolated as described above and cDNA conversion was performed using SuperScript IV Reverse Transcriptase (Thermo Fisher Scientific, USA) according to the manufacturer's instructions. Primer sequences for target genes are provided in Table 4. qPCR was performed using LightCycler®480 SYBR Green I Master (Roche, Switzerland) and data were collected using a LightCycler® 480 II (Roche). Technical triplicates were performed and results were normalized against GAPDH expression to obtain an average of relative gene expression.

TABLE 4
Primer sequence for target genes

| | SEQ | Primer Sequence in 5′-3′ |
|---|---|---|
| Targets | ID NO. | orientation |
| CCL5-Forward | 36 | ACACACTTGGCGGTTCTTTC |
| CCL5-Reverse | 37 | CCTGCTGCTTTGCCTACATT |
| CXCL16-Forward | 38 | CTACACGAGGTTCCAGCTCC |
| CXCL16-Reverse | 39 | CAATCCCCGAGTAAGCATGT |
| GAPDH-Forward | 40 | ACCACAGTCCATGCCATCAC |
| GAPDH-Reverse | 41 | TCCACCACCCTGTTGCTGTA |

### Prediction Modelling Algorithm

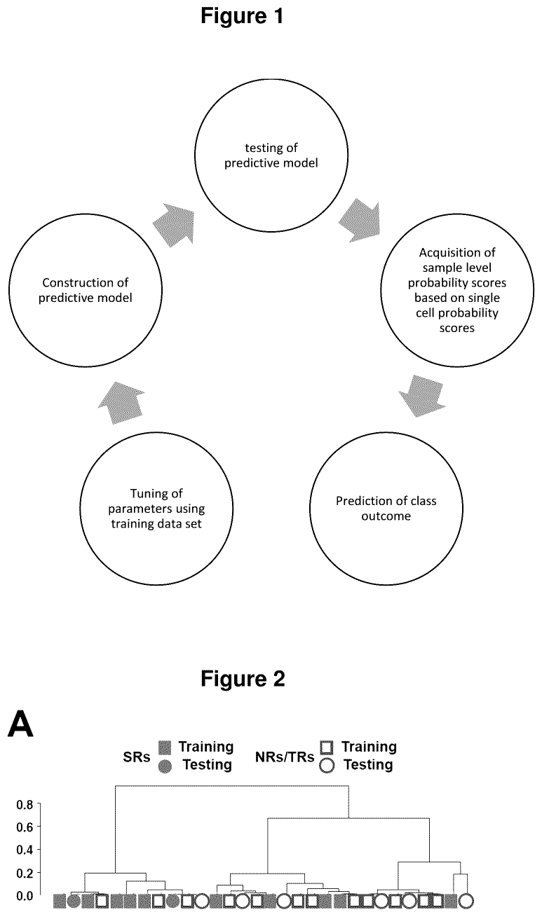

Random Forest algorithm (Breiman, Machine Learning 45, 5-32 (2001)) was used for building the model to predict the clinical response to Y90 RE. Single-cell CyTOF data (10,000 single cells with 37 markers expressions) from each patient from n=22 randomly selected patients was used for algorithm tuning and training and then tested on an independent validation or testing cohort of n=8 patients (FIG. 1). Caret (K. M, Journal of Statistical Software 28, (2008)) and Ranger (Wright, Journal of Statistical Software 77, (2017).) packages in R were used in the tuning and training of the random forest based on two parameters: mtry, the number of random variables in each tree, and ntree, the number of trees, for optimal accuracy (mtry=10 and ntree=2000 which provided maximum accuracy of 76.8% was chosen to tune the model-FIG. 2). For each of the samples, percentage of cells being classified as SR or NR was computed and used as a voting system for probability scores, where ≥50% as SR classified the samples in SR group while <50% as SR classified them in NR group. The results were compared with actual clinical outcomes and true- and false positive rates were plotted on a Receiver-Operating Characteristic (ROC) curve.

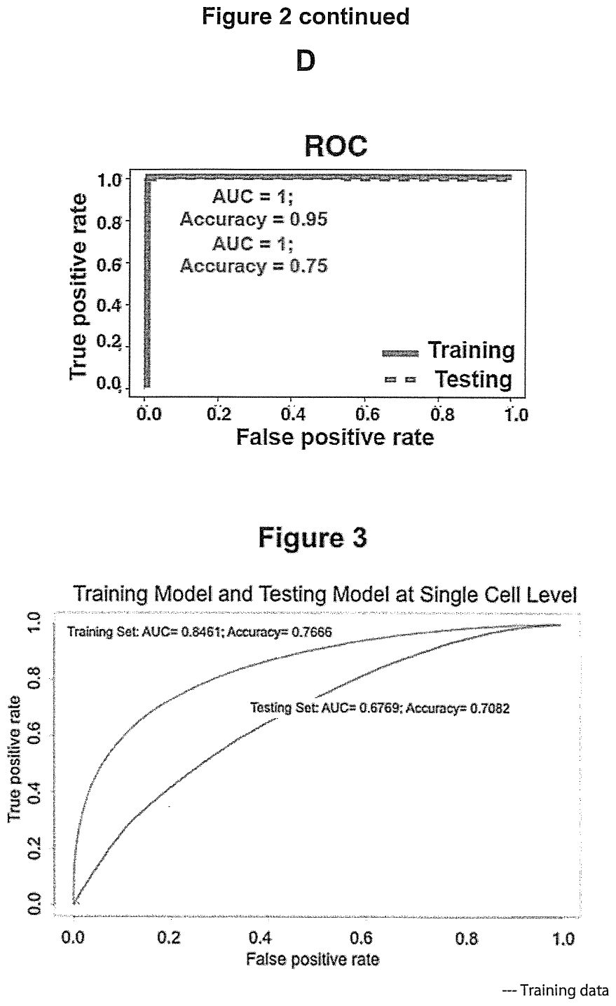

Accuracy obtained for training and testing data at single cell level are 0.7686 and 0.7082 respectively (FIG. 3), with Area under the Curve (AUC) values of 0.846 and 0.677 respectively. For the training data, this means that there is 84.6% more likely that the training model will rank a randomly chosen positive R class single cell data higher than a randomly chosen negative class NR single cell data and 76.9% of the single cell data in the training dataset are correctly classified, as reference to the actual clinical outcomes demonstrate. In the testing data set, 70.8% of the single cell data are correctly classified. Sample level probability scores are then acquired using the simple cell level probability scores (FIG. 1, Tables 5 and 6). For each of the samples in the training or testing/validation cohort, number of cells being classified as class SR and class NR are computed. Percentages of cells being classified as class SR and class NR for each sample are computed (FIG. 1, Tables 5 and 6) and are used as a voting system to be treated as probability scores for class SR and class NR at whole sample level. In this final step, in order for a sample to be classified as class SR, the probability score of positive class SR for a sample data has to be equal to or greater than 50%, without which the sample will be classified as class NR. This final step of voting system generates highly accurate prediction outcomes, which gave an accuracy of 0.955 for the training data set and 0.750 for the testing data set. Both of the training and testing data sets obtained AUC value of 1 under the ROC curve (FIG. 2D). This means that, 95.5% of the training set samples are correctly classified, and there is 100% probability that the training model will rank a randomly chosen positive responder SR class sample higher than a randomly chosen negative class NR sample. This has the clear advantage that there is a high chance that all the NR class will be treated with the relevant composition comprising a SIRT and an immunotherapy and even where an SR is predicted to be an NR where treatment with SIRT alone would be sufficient the combined treatment of a composition comprising a SIRT and an immunotherapy should still result in a response. The dendrogram showing distance between samples in both training and testing/validation cohort is depicted in FIG. 2A. Median expression values for individual markers are used.

TABLE 5
Prediction outcome for training cohort n = 22

| | No of cells | No of cells | Prob_{—} | Prob_{—} | | |
|---|---|---|---|---|---|---|
| | predicted | predicted | SR | NR | Pre- | Ac- |
| Pat ID | as SR | as NR | (%) | (%) | dicted | tual |
| HEP011 | 1502 | 8498 | 15.02 | 84.98 | NR | NR |
| HEP053 | 2214 | 7786 | 22.14 | 77.86 | NR | NR |
| HEP056 | 8892 | 1108 | 88.92 | 11.08 | SR | SR |
| HEP099 | 920 | 9080 | 9.2 | 90.8 | NR | NR |
| HEP128 | 6103 | 3897 | 61.03 | 38.97 | R | R |
| HEP131 | 9488 | 512 | 94.88 | 5.12 | R | R |
| HEP147 | 8671 | 1329 | 86.71 | 13.29 | R | R |
| HEP161 | 373 | 9627 | 3.73 | 96.27 | NR | NR |
| HEP167 | 2051 | 7949 | 20.51 | 79.49 | NR | NR |
| HEP188 | 589 | 9411 | 5.89 | 94.11 | NR | NR |
| HEP201 | 6335 | 3665 | 63.35 | 36.65 | R | R |
| HEP205 | 2437 | 7563 | 24.37 | 75.63 | NR | NR |
| HEP210 | 5944 | 4056 | 59.44 | 40.56 | R | R |
| HEP242 | 5606 | 4394 | 56.06 | 43.94 | R | R |
| HEP258 | 2659 | 7341 | 25.59 | 73.41 | NR | NR |
| HEP279 | 1197 | 8803 | 11.97 | 88.03 | NR | NR |
| HEP281 | 3211 | 6789 | 32.11 | 67.89 | NR | R |
| HEP286 | 5150 | 4028 | 56.11 | 43.89 | R | R |
| HEP291 | 855 | 9145 | 8.55 | 91.45 | NR | NR |
| HEP292 | 755 | 9245 | 7.55 | 92.45 | NR | NR |
| HEP298 | 5550 | 4023 | 57.98 | 42.02 | R | R |
| HEP313 | 1256 | 8744 | 12.56 | 87.44 | NR | NR |

TABLE 6
Prediction outcome for testing cohort n = 8

| | No of cells | No of cells | Prob_{—} | Prob_{—} | | |
|---|---|---|---|---|---|---|
| | predicted | predicted | SR | NR | Pre- | Ac- |
| Pat ID | as SR | as NR | (%) | (%) | dicted | tual |
| HEP022 | 1103 | 8897 | 11.03 | 88.97 | NR | NR |
| HEP023 | 2873 | 7127 | 28.73 | 71.27 | NR | NR |
| HEP266 | 808 | 9192 | 8.08 | 91.92 | NR | NR |
| HEP272 | 3500 | 6500 | 35 | 65 | NR | NR |
| HEP278 | 1241 | 8759 | 12.41 | 87.59 | NR | NR |
| HEP294 | 1930 | 8070 | 19.3 | 80.7 | NR | NR |
| HEP179 | 4604 | 5396 | 46.04 | 53.96 | NR | SR |
| HEP316 | 3506 | 6494 | 35.06 | 64.94 | NR | SR |

The single cell training model and sample based voting system, provides highly accurate prediction of response to SIRT Y90 RE than any other clinical parameters.

### Prediction Model for Sustained Response Based on the Immune Profiles of Pre-Y90 PBMCs

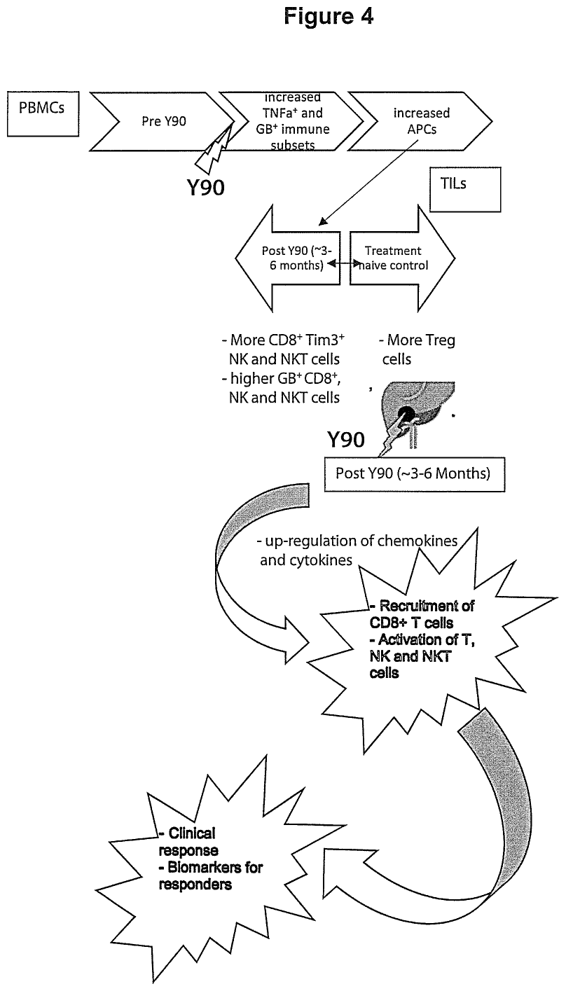

Based on the differences in immune markers expression from the peripheral blood, the patients who demonstrated sustained response from the patients who showed no or transient response to Y90-RE were segregated. Next, a prediction model was built using Random Forests for predicting sustained response based on single-cell immune profiles (CyTOF data from 10,000 single cells with 37 markers expressions) of pre-Y90 PBMCs (FIG. 2A). The prediction model was selected based on the parameters: mtry (number of random variables) and ntree (number of trees) that provided the optimal accuracy (mtry=10 and ntree=2000 provided maximum accuracy of 76.8%, FIG. 2B). A high accuracy of 95.5% was found when cross-validating this model in the training cohort (n=22) and accuracy of 75.0% when independently tested in a testing cohort (n=8) (FIG. 2D and table 5 and 6). Taken together, this in-depth immunophenotyping approach has demonstrated the nature of the immune response after Y90-RE at the local and systemic level. Potential systemic biomarkers have been identified that classify and predict HCC patients who showed sustained response to Y90-RE (FIG. 4).

### Multivariate Analysis of Variance

In order to consider other clinical parameters potentially influencing the clinical outcome or response to Y90-RE, multivariate analysis of variance was performed to analyze the relationships between the actual clinical response with the prediction model described herein as well as other clinical parameters which may influence outcome/response. For Multivariate Analysis of Variance (Manova—Xu and Cui. Bioinformatics 2008; 24:1056-62), the F-value and p-value were calculated based on Pillai-Bartlett trace statistic (Muller and New. J Comput Graph Stat 1998; 7:131-7) These parameters include: Stage, tumor multiplicity, tumor size, portal vein tumor thrombus (PVTT), alpha-fetoprotein (AFP) level, Hepatitis status and pre or post therapy prior or after Y90-RE (Table 7).

TABLE 7
Clinical and samples collection information for HCC patients treated with Y90-RE-1

| | | | | | | | 3 m | 6 m | Location of | Response |
|---|---|---|---|---|---|---|---|---|---|---|
| No. | Pat ID | Pre | 1 mo | 3 mo | 6 mo | TIL | RECIST 1.1 | RECIST 1.1 | progressi [symbol] | status |
| 1 | HEP011 | [symbol] | [symbol] | [symbol] | [symbol] | [symbol] | [symbol] | PD | Non target | NR |
| | | | | | | | | | (Distant) | |
| 2 | HEP023 | [symbol] | [symbol] | [symbol] | [symbol] | [symbol] | [symbol] | PD | Non target | NR |
| | | | | | | | | | (Distant) | |
| 3 | HEP053 | [symbol] | [symbol] | [symbol] | [symbol] | [symbol] | [symbol] | [symbol] | Target | TR |
| 4 | HEP099 | [symbol] | [symbol] | [symbol] | [symbol] | [symbol] | [symbol] | [symbol] | Non target | NR |
| | | | | | | | | | (Distant) | |
| 5 | HEP167 | [symbol] | [symbol] | [symbol] | [symbol] | [symbol] | [symbol] | [symbol] | Non target | NR |
| | | | | | | | | | (Distant) | |
| 6 | HEP205 | [symbol] | [symbol] | [symbol] | [symbol] | [symbol] | [symbol] | [symbol] | Non target | NR |
| | | | | | | | | | (Distant) | |
| 7 | HEP278 | [symbol] | [symbol] | [symbol] | [symbol] | [symbol] | [symbol] | [symbol] | Non target | TR |
| | | | | | | | | | (liver) | |
| 8 | HEP272 | [symbol] | [symbol] | [symbol] | [symbol] | [symbol] | [symbol] | [symbol] | Target | NR |
| 9 | HEP022 | [symbol] | [symbol] | [symbol] | [symbol] | [symbol] | [symbol] | [symbol] | Non target | TR |
| | | | | | | | | | (Distant) | |
| 10 | HEP279 | [symbol] | [symbol] | [symbol] | [symbol] | [symbol] | [symbol] | [symbol] | Non target | TR |
| | | | | | | | | | (liver) | |
| 11 | HEP161 | [symbol] | [symbol] | [symbol] | [symbol] | [symbol] | [symbol] | [symbol] | Target | NR |
| 12 | HEP188 | [symbol] | [symbol] | [symbol] | [symbol] | [symbol] | [symbol] | [symbol] | Non target | NR |
| | | | | | | | | | (Distant) | |
| 13 | HEP258 | [symbol] | [symbol] | [symbol] | [symbol] | [symbol] | [symbol] | [symbol] | Non target | NR |
| | | | | | | | | | (Distant) | |
| 14 | HEP266 | [symbol] | [symbol] | [symbol] | [symbol] | [symbol] | [symbol] | [symbol] | Non target | NR |
| | | | | | | | | | (Distant) | |
| 15 | HEP291 | [symbol] | [symbol] | [symbol] | [symbol] | [symbol] | [symbol] | [symbol] | Non target | NR |
| | | | | | | | | | (Distant) | |
| 16 | HEP292 | [symbol] | [symbol] | [symbol] | [symbol] | [symbol] | [symbol] | [symbol] | Non target | NR |
| | | | | | | | | | (Distant) | |
| 17 | HEP294 | [symbol] | [symbol] | [symbol] | [symbol] | [symbol] | [symbol] | [symbol] | Non target | TR |
| | | | | | | | | | (liver) | |
| 18 | HEP313 | [symbol] | [symbol] | [symbol] | [symbol] | [symbol] | [symbol] | [symbol] | Non target | NR |
| | | | | | | | | | (Distant) | |
| 19 | HEP128 | [symbol] | [symbol] | [symbol] | [symbol] | [symbol] | [symbol] | [symbol] | — | SR |
| 20 | HEP131 | [symbol] | [symbol] | [symbol] | [symbol] | [symbol] | [symbol] | [symbol] | — | SR |
| 21 | HEP147 | [symbol] | [symbol] | [symbol] | [symbol] | [symbol] | [symbol] | [symbol] | — | SR |
| 22 | HEP056 | [symbol] | [symbol] | [symbol] | [symbol] | [symbol] | [symbol] | [symbol] | — | SR |
| 23 | HEP179 | [symbol] | [symbol] | [symbol] | [symbol] | [symbol] | [symbol] | [symbol] | — | SR |
| 24 | HEP201 | [symbol] | [symbol] | [symbol] | [symbol] | [symbol] | [symbol] | [symbol] | — | SR |
| 25 | HEP132 | [symbol] | [symbol] | [symbol] | [symbol] | [symbol] | [symbol] | [symbol] | — | SR |
| 26 | HEP210 | [symbol] | [symbol] | [symbol] | [symbol] | [symbol] | [symbol] | [symbol] | — | SR |
| 27 | HEP242 | [symbol] | [symbol] | [symbol] | [symbol] | [symbol] | [symbol] | [symbol] | — | SR |
| 28 | HEP281 | [symbol] | [symbol] | [symbol] | [symbol] | [symbol] | [symbol] | [symbol] | — | SR |
| 29 | HEP286 | [symbol] | [symbol] | [symbol] | [symbol] | [symbol] | [symbol] | [symbol] | — | SR |
| 30 | HEP298 | [symbol] | [symbol] | [symbol] | [symbol] | [symbol] | [symbol] | [symbol] | — | SR |
| 31 | HEP316 | [symbol] | [symbol] | [symbol] | [symbol] | [symbol] | [symbol] | [symbol] | — | SR |

| | Tumor | Tumor | Stage | | AFP level | Hepatitis | Pre | Post |
|---|---|---|---|---|---|---|---|---|
| No. | multiplicit [symbol] | size (cm) | (T [symbol] ) | PVTT | (μg/ml) | status | tx | tx |
| 1 | Multifocal | 3.2 | IVB | No | 16841.0 | B | Yes | No |
| 2 | 1 | 9.1 | IIIB | Yes | 2047.0 | B | Yes | No |
| 3 | Multifocal | 1.7 | IIIB | Yes | 210.0 | B | Yes | Yes |
| 4 | Multifocal | 2.2 | IIIB | Yes | 402.0 | C | Yes | Yes |
| 5 | Multifocal | 7.2 | IIIB | Yes | 171.0 | Non | Yes | Yes |
| 6 | 1 | 10.0 | IIIA | No | 6.3 | B | No | No |
| 7 | Multifocal | 3.0 | [symbol] | No | 15.9 | Non | No | Yes |
| 8 | 1 | 16.0 | I | No | 2.3 | Non | No | No |
| 9 | Multifocal | 2.5 | [symbol] | Yes | 252.0 | B | Yes | Yes |
| 10 | Multifocal | 8.4 | IIIA | No | 202.0 | B | No | Yes |
| 11 | Multifocal | 3.7 | IIIb | Yes | 499.0 | Non | No | Yes |
| 12 | Multifocal | 10.6 | IIIB | Yes | 62.4 | Non | No | Yes |
| 13 | Multifocal | 2.7 | IIIB | Yes | 60500.0 | C | Yes | No |
| 14 | Multifocal | 8.3 | IIIA | No | 122.0 | B | No | No |
| 15 | Multifocal | 6.2 | IIIB | Yes | 3.7 | B | No | Yes |
| 16 | Multifocal | 5.7 | IIIA | No | 17818.0 | Non | Yes | No |
| 17 | 1 | 9.9 | I | No | 521.0 | B | Yes | Yes |
| 18 | Multifocal | 10.0 | IIIA | No | 4281.0 | Non | Yes | Yes |
| 19 | 1 | 10.3 | [symbol] | Yes | 97.5 | B | Yes | Yes |
| 20 | 1 | 5.5 | I | Yes | 9.9 | B | No | Yes |
| 21 | Multifocal | 9.8 | IIIA | Yes | 67.7 | Non | No | No |
| 22 | 1 | 10.0 | I | No | 4718.0 | C | Yes | Yes |
| 23 | 1 | 5.1 | IIIB | Yes | 149.0 | Non | Yes | Yes |
| 24 | Multifocal | 3.9 | [symbol] | Yes | 165.0 | C | Yes | No |
| 25 | 1 | 12.6 | I | No | 3.9 | Non | Yes | No |
| 26 | Multifocal | 6.4 | I | No | 38.0 | B | No | Yes |
| 27 | 1 | 11.1 | I | No | 14.4 | B | No | Yes |
| 28 | 1 | 6.0 | I | No | 6.4 | C | No | No |
| 29 | 1 | 6.7 | I | No | 90.2 | B | No | Yes |
| 30 | 1 | 6.4 | I | No | 3.2 | Non | No | No |
| 31 | Multifocal | 7.0 | IIIA | No | 4.0 | C | Yes | No |

[symbol]

indicates data missing or illegible when filed

[symbol]

As sho diction model described herein was superior in predict [symbol] 7) compared to stage (p=0.0012); or tumor multiple [symbol] of the other parameters which do not have signific [symbol] er. This indicated that the immune status of pre-Y90RE PBMC [symbol] as a better biomarker than other clinical parameters in predicting sustained response to Y90-RE. Accordingly the prediction model described herein is highly superior.

TABLE 8
Multivariate Analysis of Variance (Manova)

| Variables | F value | p value |
|---|---|---|
| Prediction Model | 50.4000 | 1.006e⁻⁰⁷*** |
| Tumor multiplicity | 6.8923 | 0.01387 * |
| Tumor Size | 0.2748 | 0.6042 |
| Stage | 13.0980 | 0.001155 ** |
| AFP Level | 1.5452 | 0.2242 |
| PVTT | 0.1888 | 0.6673 |
| Hepatitis Status | 0.0265 | 0.8718 |
| Pre-Y90-RE tx | 0.5283 | 0.4734 |
| Post-Y90-RE tx | 0.0216 | 0.8842 |

F-value = value calculated From F-statistic
p value =
p < 0.001***
p < 0.01 **
p < 0.05 *

### Statistical Analyses

For CyTOF data, non-parametric paired or unpaired Mann-Whitney U tests were used to identify differential nodes between the two groups. A paired or unpaired

Student's t-test or Mann-Whitney U-test and Pearson's correlation test GraphPad Prism V.6.0f) was used to analyse the FlowJo and qPCR data, as indicated.

### General Response to Y90-RE Treatment

The data obtained from analysing resected tumour tissue and PBMCs from patients with HCC, provide strong evidence that Y90-RE induces both a localized and systemic immune response that involves T cell, NK cell and NKT cell activation, antigen presentation and immune-cell motility. Systemic immune subsets unique to patients who demonstrated sustained-response to Y90-RE included, exhaustion markers (PD-1 and Tim-3)-expressing CD8+ T cells and CD4+ T cells and homing receptors (CCR5 and CXCR6)-expressing CD8+ T cells and CD8+ Tim3+ T cells. A chemotaxis pathway triggered by Y90-RE for activated GB+CD8+ T cells via CCL5 and CXCL16 was also discovered. Importantly, the current study provided a prediction model for sustained clinical response based on the immune profiles of the pre-Y90-RE PBMCs.

In-depth immunophenotyping of TILs showed marked immune activation in the local tumour microenvironment, such as an increase in activated or GB-expressing-CD8+ T cells, -CD56+NK cells and -CD8+CD56+ NKT cells and reduced TREG cells post-Y90-RE. Analysis of NGS data from tumour tissues also provided compelling evidence for enhanced T cell, NK cell and NKT cell activation. For instance, in patients that received Y90-RE, an induction of CD28 co-stimulatory and CD28-dependent Vav1 and Akt pathways was observed, which have been previously shown to positively regulate T-cell activation and proliferation (Charvet, The Journal of Immunology 177, 5024-5031 (2006)). The CyTOF and NGS analyses also identified an enhanced innate immune response as a result of Y90-RE that involved NK cells and NKT cells activation.

### Y90-RE Activates the Local Immune Response

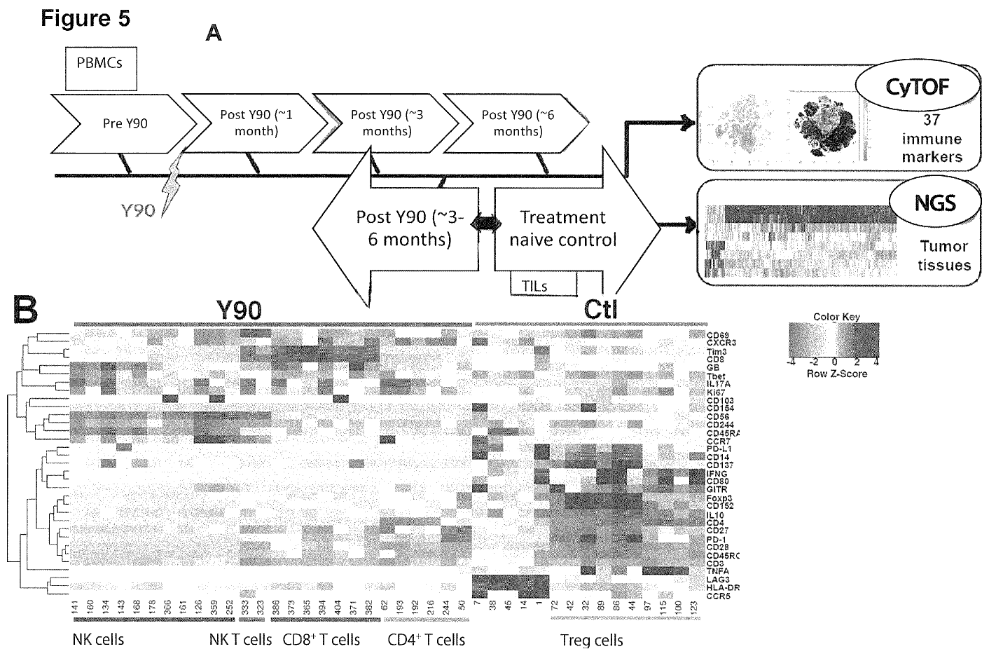

An in-depth analysis pipeline based on CyTOF and NGS was designed to survey the immune phenotypes of TILs, tumour tissues and PBMCs obtained from patients with HCC before and after undergoing Y90-RE (FIG. 5A). In order to understand the nature of the local immune response, TILs were isolated from patients after Y90-RE or from patients who were treatment-naive (with matched clinical parameters, as control, Ctl, Table 1). TILs were analysed using CyTOF (Table 3) and the differentially expressed nodes/immune-subsets from post-Y90-RE versus Ctl tumors were identified (FIG. 5B).

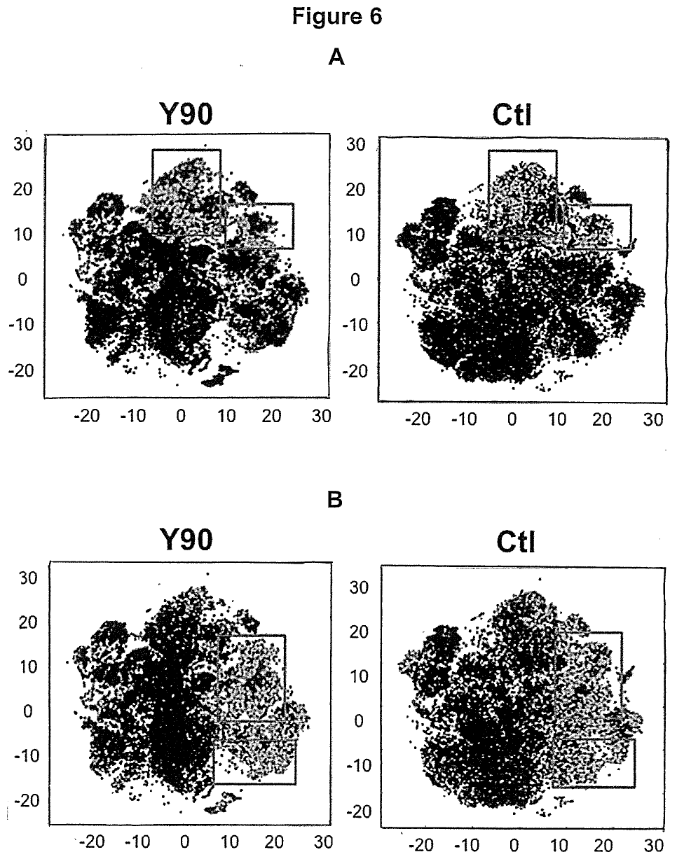

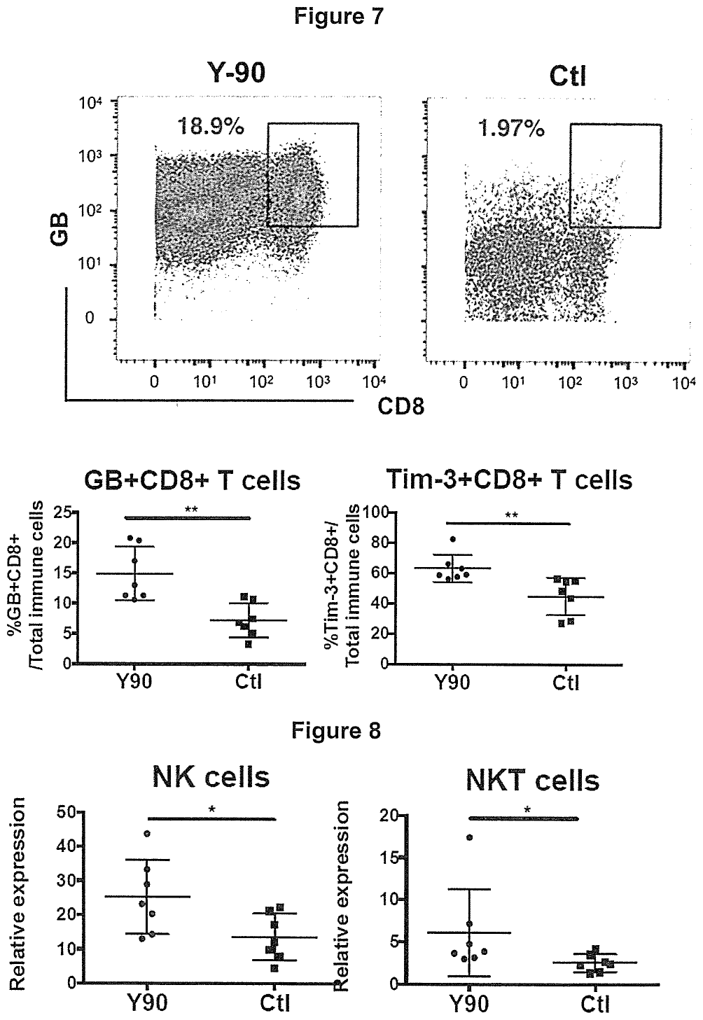

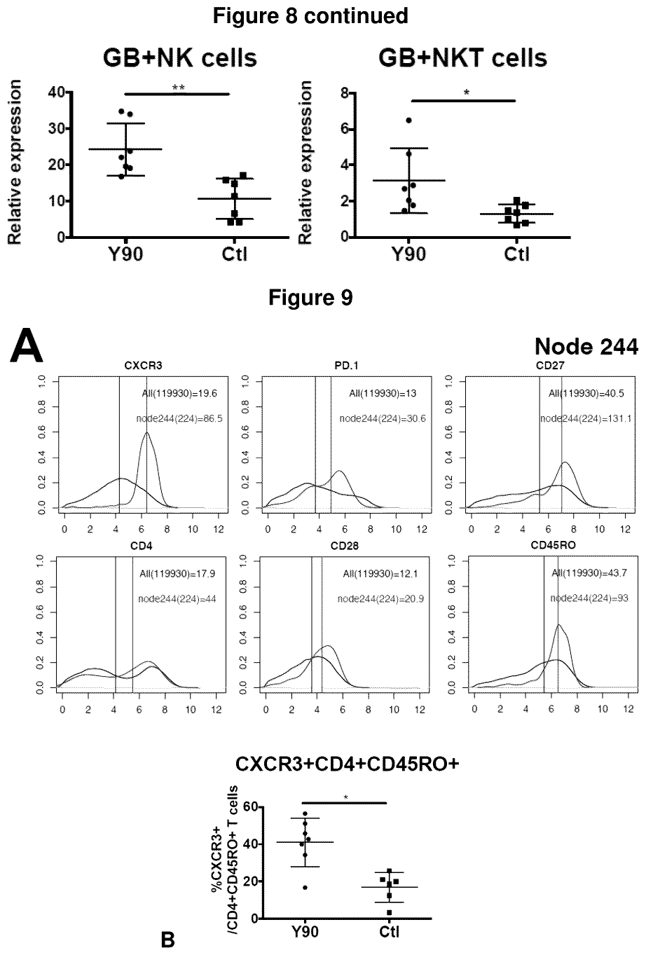

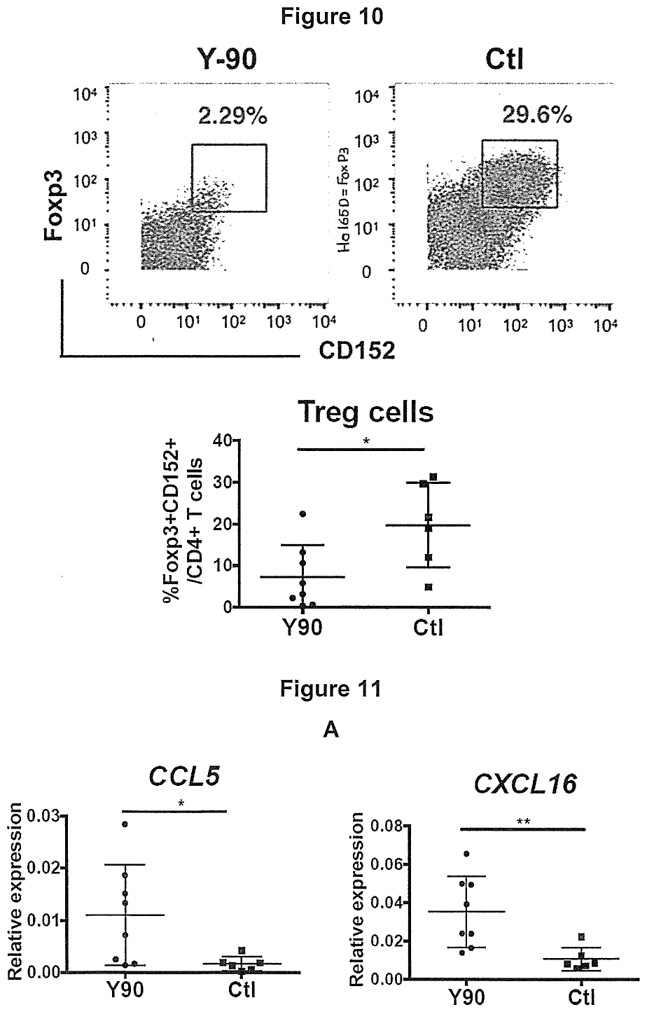

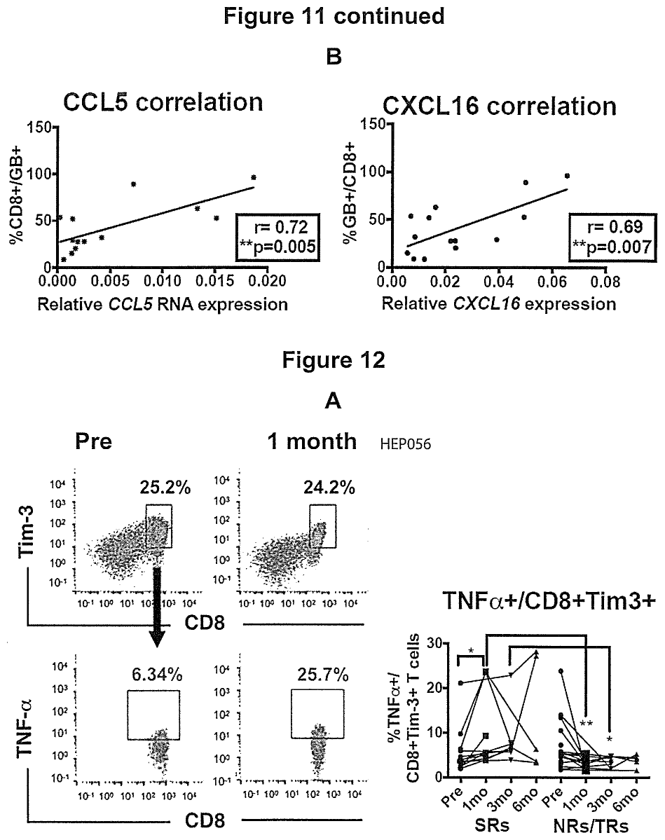

An enrichment of specific CD56+NK cells, CD8+CD56+ NKT cells, CD8+ T cells and CD4+ T cell subsets in TILs isolated from post-Y90-RE tumours was observed (FIG. 5B). The expression of two immune markers, granzyme B (GB) and Tim-3, was higher in TILs from post-Y90-RE versus Control (Ctl) tumours (FIG. 6A & B). Indeed, a higher percentage of GB+CD8+ T cells and Tim-3+CD8+ T cells were detected in post-Y90-RE TILs as confirmed using FlowJo manual gating (FIG. 7). An overall enrichment of CD56+NK cells and CD8+CD56+ NKT cells, which expressed significantly more GB than the Ctl TILs, was also observed in post-Y90-RE TILs (FIG. 8). In addition, a higher percentage of CD4+CD45RO+ T cells that expressed CXCR3 in post Y90-RE-TILs was observed (FIG. 5B and FIG. 9). Conversely, Ctl TILs showed a higher percentage of Foxp3+CD152+CD4+ TREG cells (FIG. 5B and FIG. 10) as compared to Y90-RE TILs. Taken together, these data show that the immune microenvironment of post-Y90-RE tumours was infiltrated by multiple activated immune subsets and was less immunosuppressive compared to the TREG cells-enriched Ctl tumours.

Immune Activation Pathways are Induced in the Tumor Tissue Following Treatment with Y90-RE

NGS was performed on the resected tumour tissues collected from HCC patients downstaged after Y90-RE comparing to patients who did not receive any prior treatment as Ctl (Table 1). Overall, it was observed that multiple differentially expressed genes, with 88% of highly expressed genes in post-Y90-RE compared to Ctl tumours. Functional analysis using DAVID pathway analysis tool found that most of these enriched genes were related to innate or adaptive immune responses. Conversely, the genes enriched in Ctl tumours were not related to immune pathways.

Further data analysis of these post Y90-RE-enriched genes using the Reactome database identified pathways including the antigen presentation of MHC class II molecule; T-cell activation pathways, that were related to the CD28 co-stimulatory and CD28-dependent Vav1 and Akt pathways. Comparing post-Y90-RE versus Ctl tumours, upregulation of the NK cell activation pathway via CD244 and CD48 was also detected, as well as enrichment of LFA-1 and ICAM-1 binding, which is required for the development and recruitment of NKT cells to the liver. Taken together, these findings complement the observations by CyTOF of an enhanced activation and recruitment of T-NK- and NKT-cells into post-Y90-RE tumours.

### Y90-RE Induces Chemotaxis of CD8+ T Cells to the Tumor Microenvironment

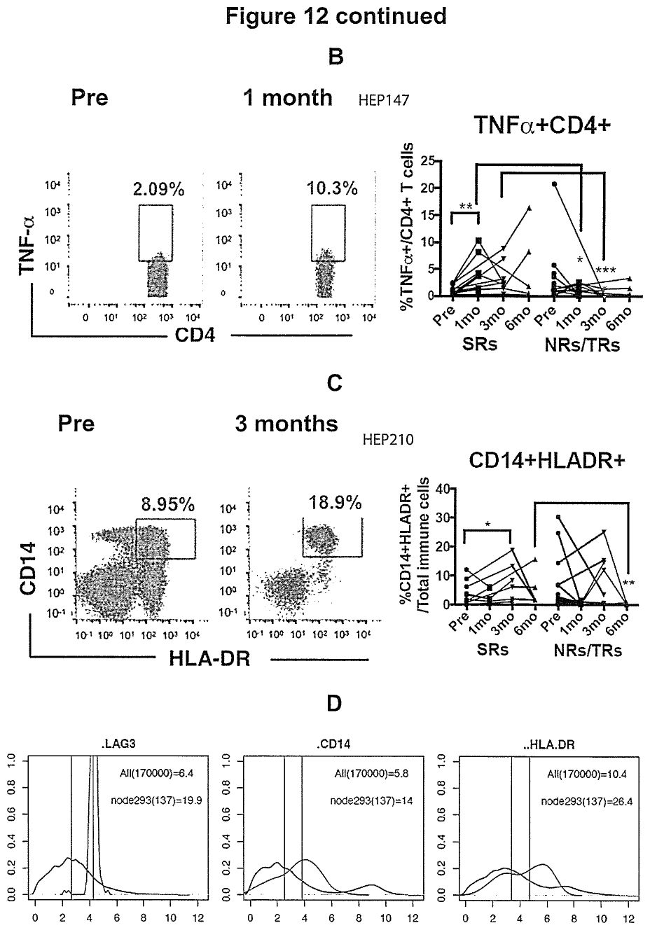

Reactome analysis on post-Y90-RE-enriched genes also indicated an increase in chemotactic activity involving the up-regulation of CXCL16 and CCL5 (FIG. 11). Given this result, it was hypothesized that a chemotaxis pathway may be induced by Y90-RE.

qPCR was then performed on tumour samples (Table 1) obtained from the same patients to validate the NGS results, which indeed showed an increase in CCL5 and CXCL16 expression-two chemokines that bind CCR5 and CXCR6, respectively (FIG. 11A). In order to confirm their chemotactic effect for activated T cells, the RNA expression of CCL5 and CXCL 16 was correlated with the immune subsets found in TILs and confirmed that CCL5 and CXCL16 were positively correlated with percentage of activated GB+CD8+ T cells (FIG. 11B). These findings demonstrated the ability of Y90-RE to shape the microenvironment of HCC tumours, not only by inducing tumour-cell death but T-cell recruitment and activation following therapy.

### Early and Late Immune Responses are Induced by Y90 Radioembolization

In order to capture the Y90-RE-induced systemic immune response, PBMCs from another 31 HCC patients were collected before and at various time points (1, 3 and 6-months) after Y90-RE (Table 2).

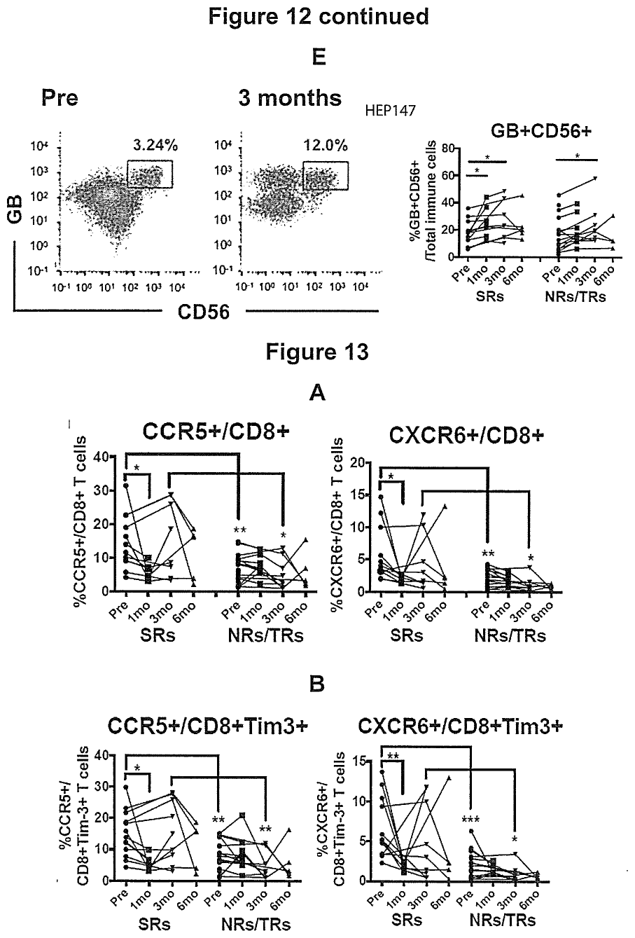

The 31 patients who received Y90-RE were segregated into two groups-Sustained responders (SRs) and non- or transient-responders (NRs/TRs) (Table 2; SRs are patients without progressive disease at any site (Non-PD) at 6 months after Y90 RE; NRs are patients who did not show even stable disease (SD) at 3 months and TRs are patients who showed initial response at 3 months but progressed by 6 months) and performed paired-wise time-points CyTOF analyses specifically on the SRs (FIG. 4). Initial indications of immune activation were represented by an increase in TNFα expression on CD8+ Tim3+ and CD4+ T cells 1-month after Y90-RE, specifically in the SRs (FIG. 12A and FIG. 12B). Notably, TNFα expression on these T-cell subsets was significantly higher in SRs versus NRs/TRs at 1- and 3-months after therapy (FIG. 12A and FIG. 12B).

The same comparisons were made between 3-months and pre-Y90-RE and a significantly higher proportion of CD14+HLADR+ antigen presenting cells (APCs) was observed 3-months after therapy specifically in SRs (FIG. 12C and FIG. 12D). GB+CD56+NK cells were also significantly enhanced at 3-months but this elevation was only specific to SRs at 1-month post-Y90-RE (FIG. 12E). A less distinct difference in immune-cell subsets was identified when comparing PBMCs at 6-months with pre-Y90-RE except APCs (FIG. 12C).

### Prediction of Response to Y90_RE Treatment

Analysis of PBMCs before and after Y90-RE by CyTOF allowed us to capture the systemic immune response triggered by the therapy and identify potential biomarkers to predict clinical outcome. The immune response that was first detected at 1-month post-Y90-RE was an increase in cytokine TNF expression on CD8+ and CD4+ T cells, followed by increased APCs 3-months post-Y90-RE (FIG. 12). The high percentages of systemic CD8+ T cells and CD4+CD45RO+ T cells that expressed PD-1 and Tim-3 before Y90-RE denoted the patients who went on to elicit a sustained response after therapy with an increase in time-to-tumour-progression of >6 months. The same immune subset, CD8+ Tim-3+ T cells, was also one of the key subsets enriched in TILs in tumours from post-Y90-RE (FIG. 7).

Tim-3 is a marker of immune-cell exhaustion and is associated with the progression of various cancers, including HCC (Li, et al. Hepatology 56, 1342-1351 (2012)). Co-expression of PD-1 and Tim-3 can enhance T cell impairment and is also associated with tumour progression (Fourcade et al. J Exp Med 207, 2175-2186 (2010)). However, Tim-3 expression is also an indication of prior T cell activation (Hastings et al. Eur J Immunol 39, 2492-2501 (2009)), and indeed, the co-expression of Tim-3 and PD-1 on CD8+ T cells may indicate a prior immune response mounted towards tumour antigens. Another indication of heightened T cell activation was the co-expression of pro-inflammatory GB and TNFα on TILs or PBMCs in patients after Y90-RE (FIG. 7 and FIG. 12A). Targeting both Tim-3 and PD-1 pathways can reverse T cell exhaustion and restore anti-tumour immune responses in murine cancer models (Sakuishi, et al. J Exp Med 207, 2187-2194 (2010)). Given these data, it is postulate that sequential therapy involving Y90-RE followed by immunotherapy using check-point inhibitors against the PD-1/PD-L1 or Tim-3 pathways, particularly in the sustained-responders, may enhance the clinical response in HCC.

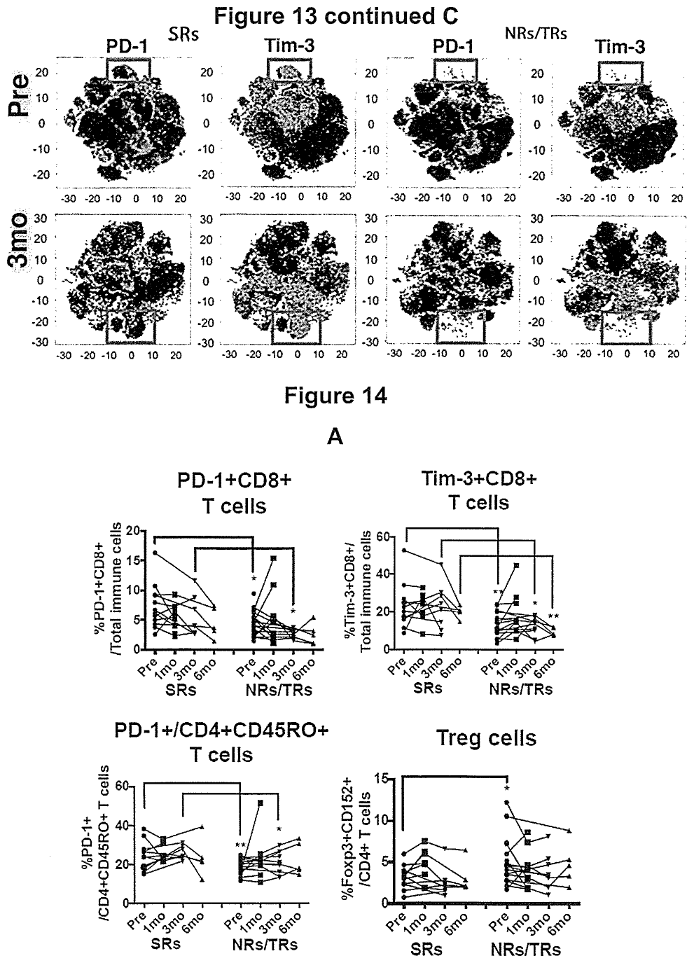

The co-expression of Tim-3 with the homing receptors CCR5 and CXCR6 on CD8+ T cells indicated their ability to home towards CCL5 and CXCL16 chemokines. The data demonstrated that CCR5+CD8+ and CXCR6+CD8+ or CCR5+/CXCR6+ Tim-3+CD8+ T cells are other biomarkers of responders to Y90-RE (FIG. 13A).

Interestingly, NGS and qPCR indicated an up-regulation of CCL5 and CXCL16 in tumour microenvironment of post-Y90-RE (FIG. 11). These results imply that Y90-RE-induced chemokine expression is required for the recruitment of cytotoxic CD8+ T cells to tumour sites via CCL5 and CXCL16 pathways, and that this effect leads to a clinical response to Y90-RE (FIG. 11B).

Deep immunophenotyping and transcriptomic analysis demonstrated robust immune activation locally within the tumour microenvironment and systemically in the peripheral blood of HCC patients who showed sustained-response to Y90-RE. By this approach the immune activation was captured and predictive biomarkers were identified for sustained clinical response in the peripheral blood that may guide treatment choices for HCC patients.

### Higher Expression of PD-1 and Tim-3 Identifies and Predicts Sustained Response to Y90_RE

The systemic immune profiles of SRs versus NRs/TRs were compared at pre- and 3-months after Y90-RE. Distinct CD4+ T cells and CD8+ T-cell specific to SRs were observed. PD-1 and Tim-3 showed higher expression in SRs both pre- and 3-months after Y90-RE (FIG. 13B).

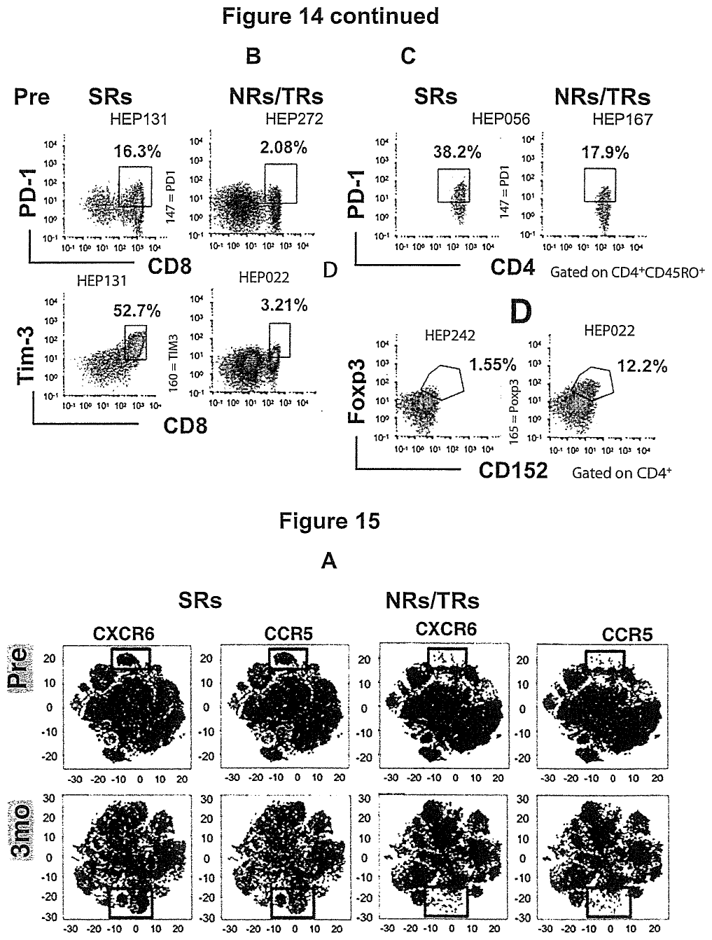

Validated by FlowJo manual gating, the higher percentages of PD-1- and Tim-3-expressing CD8+ T cells were shown in SRs at both time points (FIG. 14A and FIG. 14B). A higher percentage of PD-1+CD4+CD45RO+ T cells in SRs before and 3-months after therapy was also observed (FIG. 13A and FIG. 14C). Of note, Tim-3+CD8+ T cells remained higher in SRs up to 6-months after Y90-RE (FIG. 14A).

This apparent higher expression of exhaustion markers may indicate a higher level of T-cell activation that is specific to SRs both prior to and 3 or 6-months after the therapy. This may mediate in part the subsequent sustained response to Y90-RE. In contrast, the immune subsets enriched in the NRs/TRs at pre- or 3 months post-Y90-RE included CD4+Foxp3+CD152+ Treg (FIG. 14B and FIG. 14D); CD4+CD45RO+ T cells that do not express PD-1 and CD8+ T cells that do not express PD-1 or Tim-3.

T Cells from Sustained Responders of Y90-RE Express Specific Homing Receptors

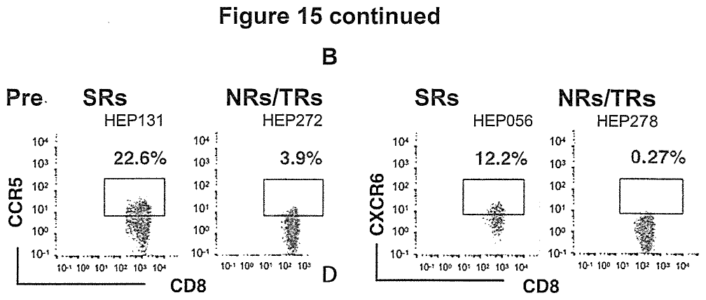

Distinct differences in chemokine-receptor expression, namely CCR5 and CXCR6, were observed when comparing SRs with NRs/TRs pre- and 3-months post-Y90-RE (FIG. 15A). FlowJo manual gating on all samples confirmed a significantly higher percentages of CCR5+CD8+ T cells and CXCR6+CD8+ T cells from SRs at both time points (FIG. 13A and FIG. 15B). Interestingly, the expression of CCR5 and CXCR6 was also significantly higher on the Tim-3+CD8+ T-cell subsets that were enriched in SRs (FIG. 14A and FIG. 14B). The higher percentages of CCR5- and CXCR6-expressing CD8+ T cells is also consistent with the previous results showing the recruitment of activated CD8+ T cells to the tumour microenvironment upon Y90-RE (FIG. 11B). Furthermore, a drop in percentage of both CCR5 and CXCR6 on CD8+ T cells and CD8+ Tim-3+ T-cells that was observed at 1-month post-Y90-RE further implicated the recruitment of activated T cells to the tumour site upon Y90-RE (FIG. 13A).

It should be further appreciated by the person skilled in the art that variations and combinations of features described above, not being alternatives or substitutes, may be combined to form yet further embodiments falling within the intended scope of the invention.

What is claimed is:

1. A system for prognosing a response to treatment for a patient suffering from cancer, the system comprising:
a processing unit operable to:
obtain a dataset of immune marker expression profiles in a leukocyte sample taken from the patient with cancer;
sort the dataset based on a probability that an immune marker expression of a plurality of cells in the leukocyte sample will correspond to patients with cancer that respond to radioembolization (RE) or not respond to or only transiently respond to RE; and
allocate a probability score to the sample taken from the patient with cancer that the patient will (i) respond to RE or (ii) not respond or only transiently respond to RE treatment, wherein the probability score of the sample is obtained from the majority of the plurality of cells having the immune marker expression correspond to patients with cancer that respond to RE or correspond to patients with cancer that do not respond or transiently respond to RE.

2. The system of claim 2, further comprising a device for measuring expression of immune markers in a leukocyte sample taken from the patient with cancer.

3. The system of claim 2, further comprising a device for measuring expression of immune markers in a leukocyte sample taken from the patient with cancer wherein the device is a cytometer a next generation sequencer or a thermo cycler.

4. A method of treating a patient with cancer that is prognosed as a sustained responders (SR) to radioembolization (RE) comprising administering a therapeutically effective dose of a composition comprising a RE and an immunotherapy to the patient.

5. The method of claim 4, wherein the RE is Y90-RE.

6. The method of claim 4, wherein the cancer is hepatocellular carcinoma.
